(19) United States
(12) Patent Application Publication
(10) Pub. No.: US 2011/0231942 A1
(43) Pub. Date: Sep. 22, 2011

(54) TIKI1 AND TIKI2, WNT INHIBITORS

(76) Inventors: Xi He, Jamaica Plain, MA (US); Chika Yokota, Stockholm (SE); Xinjun Zhang, Brookline, MA (US); Bryan MacDonald, Brookline, MA (US)

(21) Appl. No.: 13/049,436

(22) Filed: Mar. 16, 2011

Related U.S. Application Data
(60) Provisional application No. 61/314,885, filed on Mar. 17, 2010.
(60) Provisional application No. 61/363,025, filed on Jul. 9, 2010.

(51) Int. Cl. A61K 38/17; C07K 14/435; C07H 21/00; C07K 16/00; A61K 31/7088; A61K 31/713; A61P 19/10; A61P 35/00; C12Q 1/02; C12N 15/63; C12N 1/00; C12N 5/10; A01K 67/00

(52) U.S. Cl. 800/13; 530/350; 536/23.5; 530/387.9; 514/21.2; 514/44 R; 514/19.3; 514/44 A; 435/29; 435/320.1; 435/243; 435/325

(57) ABSTRACT

This invention relates to Tiki1 and Tiki2 proteins and nucleic acids, cells expressing the same, and methods for identifying compounds that modulate Tiki1/2 activity for use in the treatment of osteoporosis or cellular proliferative disorders.

### CLAIM OF PRIORITY

This application claims the benefit of U.S. Provisional Application Ser. No. 61/314,885, Mar. 17, 2010, and U.S. Provisional Application Ser. No. 61/363,025, filed on Jul. 9, 2010, which are incorporated herein by reference in their entirety.

### FEDERALLY SPONSORED RESEARCH OR DEVELOPMENT

This invention was made with Government support under Grant No. GM57603 awarded by the National Institutes of Health. The Government has certain rights in the invention.

### TECHNICAL FIELD

This invention relates to Tiki1 and Tiki2 proteins and nucleic acids, cells expressing the same, and methods for identifying compounds that modulate Tiki1/2 activity for use in the treatment of osteoporosis and other skeletal diseases, e.g., arthritis, or cellular proliferative disorders.

### BACKGROUND

Osteoporosis is a global health issue, particularly as the population ages. More than 200 million people worldwide suffer from osteoporosis, and three to four times more are at risk of osteoporosis due to low bone mass (Kanis et al. Bone. 2008. 42:467-75). Bone-related injuries in the general including pediatric population present additional huge medical burden. Identification of new therapeutic strategies for treating osteoporosis and promoting bone regeneration has vast medical, societal and economical implications.

### SUMMARY

Tiki1 and Tiki2 are Wnt inhibitors. They inhibit the Wnt signaling and responsive reporter expression, likely by inactivating Wnt proteins through deacylation or depalmitoylation. They are transmembrane proteins, and are unique among all Wnt antagonists (secreted); they down-regulate Wnt signaling in both Wnt-producing and Wnt-responsive cells. They are excellent targets for therapies in cancers and osteoporosis.

Thus, in one aspect, the invention provides methods for identifying a candidate compound for maintaining or increasing bone density or for treating a cellular proliferative disorder. The methods include providing a cell that is responsive to Wnt3a that expresses Tiki1 or Tiki2; treating the cell with a test compound; evaluating an effect of the test compound on signaling in response to Wnt3a in the cell, and identifying as a candidate compound a test compound that relieves Tiki1 or Tiki2 inhibition of signaling in response to Wnt3a in the cell.

In some embodiments, the methods further include determining that the candidate compound acts specifically on Tiki1 or Tiki2.

In additional aspects, the invention provides isolated nucleic acids encoding Tiki1N or Tiki2N, e.g., Human, Frog, Fish or Worm Tiki1N or Tiki2N; and vectors including the nucleic acids. In some embodiments, the vectors further include one or more regulatory sequences operatively linked to the Tiki1N/2N nucleic acid sequence. Also provided are host cells expressing the vectors or nucleic acids.

In another aspect, the invention provides isolated Tiki1N or Tiki2N polypeptides, e.g., recombinant polypeptides.

A Tiki1N or Tiki2N polypeptide is one that lacks the C-terminal transmembrane domain, e.g., in which the C-terminal 23 or 24 amino acids were removed, e.g., as shown in FIG. 12, or residues corresponding thereto as shown in FIG. 1.

In a further aspect, the invention provides antibodies, e.g., monoclonal antibodies, or antigen binding fragments thereof that specifically bind to a Tiki1 or Tiki2 polypeptide.

In yet another aspect, the invention provides transgenic (knock-in) animals, e.g., rodents, e.g., mice, having an exogenous recombinant Tiki1 or Tiki2 nucleic acid molecule stably integrated into the genome of said mammal.

In yet another aspect, the invention provides transgenic (knock-out) animals, e.g., rodents, e.g., mice, whose somatic and germ cells comprise a disrupted Tiki1 or Tiki2 gene.

Also provided herein are cells derived from these transgenic animals.

In another aspect, the invention features methods for treating a disorder associated with increased Wnt signaling, e.g., with unwanted cellular proliferation, e.g., cancer. The methods include administering a therapeutically effective amount of a composition comprising: (ii) Tiki1 or Tiki2 polypeptide, or a variant or active fragment thereof; (ii) Tiki1N or Tiki2N polypeptide, or a variant thereof; (iii) a nucleic acid encoding a Tiki1 or Tiki2 polypeptide, or a variant or active fragment thereof; or (iv) a nucleic acid encoding a Tiki1N or Tiki2N polypeptide, or a variant thereof.

In another aspect, the invention provides methods for treating disorders associated with reduced Wnt signaling, e.g., with bone loss or resorption, e.g., osteoporosis, or some cancers, e.g., melanoma, the method comprising administering a therapeutically effective amount of an inhibitory nucleic acid that specifically decreases expression of Tiki1 or Tiki2.

In some embodiments, the inhibitory nucleic acid is selected from the group consisting of siRNA, antisense, ribozymes, and aptamers.

As used herein the notation “Tiki1/2” means that either or both of Tiki1 or Tiki2 could be used.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a ClustalW alignment of Tiki proteins from representative vertebrates and invertebrates. Protein sequences from NCBI: Human Tiki1 (Q86V40/LOC 129293) (SEQ ID NO:1), Human Tiki2 (XP_{—}371250/LOC388630) (SEQ ID NO:2), and C. elegans (CAA94108/C05G5.5) (SEQ ID NO:3). Other proteins from Xenopus tropicalis (TIKI1, SEQ ID NO:4; TIKI2, SEQ ID NO:5), Danio rerio (TIKI1, SEQ ID NO:6; TIKI2, SEQ ID NO:7) and Nemostella vectensis (partial; SEQ ID NO:8) were derived from genomic sequences. Black, grey, and light grey represent identical, conservative, and similar amino acid residues, respectively.

FIG. 2A is an image of three RT-PCR results. Top panel, Tiki1 inhibited Xwnt8-induced Xnr3 expression in animal caps of Xenopus embryos. 2.5 or 5 pg of Xwnt8 mRNA was injected with or without 2 ng Tiki1 mRNA at the 2-cell stage, and animal caps were dissected at stage 9 for RT-PCR. EF-1alpha was a loading control. uninj: uninjected embryos; WE: whole embryos. Middle panel, Tiki1 did not inhibit Nodal signaling in embryos. 5 or 10 pg of Xnr-1 mRNA with or without 2 ng of Tiki1 mRNA were injected at the 2-cell stage and animal caps were dissected at stage 9 for RT-PCR. Bottom panel, Tiki1 did not inhibit FGF signaling in embryos. 2 ng of Tiki1 mRNA were injected at the 2-cell stage. Animal caps were dissected at stage 8.5 from control or injected embryos, and treated with 100 or 500 ng/ml bFGF until stage 10 before RT-PCR.

FIG. 2B is a bar graph showing that Tiki1 (400 pg) inhibited axis duplication by Xwnt8 (10 pg) but not by constitutively activated LRP6ΔN (100 pg), Xdsh (1 ng) or beta-catenin (100 pg). Indicated mRNAs were injected into the ventral marginal zone at the 8-cell stage and the phenotype was scored at the tadpole stage. LDLR was used as a negative control. Numbers indicate embryos injected and scored.

FIG. 2C is a bar graph showing that Human Tiki1 and Tiki2 inhibit Wnt3a signaling in HEK293T cells using the TOPFLASH reporter assay. Wnt3a and Tiki1 or Tiki2 were co-transfected. The TOPFLASH reading (normalized to Renilla) in Wnt3a alone was set as 100. DNA transfection amounts are shown per well in a standard 24-well plate format. Representative data from one of three independent experiments is shown.

FIG. 3A is a gel showing that RT-PCR revealed that Tiki1 mRNA becomes detectable at stage 9 and is prominently expressed from stage 10 (early gastrula) to stage 30 (tadpole).

FIG. 3B is a gel showing that Tiki1 is expressed dorsally. Stage 10 embryos were cut into four parts, animal, vegetal, dorsal and ventral, and used for RT-PCR. Xnr3 serves as a positive control.

FIG. 3C is a gel showing that Tiki1 expression is regulated by maternal Wnt signaling. Whole embryos were used for LiCl or UV treatment. 20 or 40 pg of Xwnt8 mRNA was injected into ventral marginal zone (VMZ) at 4 to 8-cell stage and VMZ was dissected at stage 10.5 for RT-PCR. ΔNTCF mRNA (1 or 2 ng) was injected into dorsal marginal zone (DMZ) at 4 to 8-cell stage and DMZ was dissected at stage 10.5 for RT-PCR. Con: control.

FIG. 4A is a gel showing that Xenopus Tiki1 protein synthesis is specifically targeted by an antisense morpholino oligonucleotide (MO). 500 pg mRNA for Tiki1-FLAG (targeted by MO) or HA-Tiki1 (resistant to MO) were injected with or without the Tiki1MO (5, 10, 20, 40 ng) into the animal pole at the 2-cell stage, and animal caps were dissected at stage 10 for Western blotting.

FIG. 4B is a bar graph showing statistical data of Tiki1MO phenotype demonstrating that Xenopus Tiki1 is required for anterior development/head formation.

FIG. 5A is a gel showing that Tiki2 inhibited beta-catenin stabilization in Wnt3a-expressing cells. Wnt3a CM rescued Tiki2 inhibition of beta-catenin stabilization in Wnt3a-expressing cells. Indicated cell lines were treated with control or Wnt3a CM for 2 hrs and whole cell lysates (WCL) were subjected for Western blotting. Beta-tubulin was a loading control.

FIG. 5B is a gel showing that Wnt3a was secreted similarly in the presence or absence of Tiki2, but exhibited faster electrophoretic migration. CM or WCL were analyzed by Western blotting.

FIG. 5C is a gel showing that Wnt3a secreted from HEK293T cells in the presence of Tiki1 or Tiki2 exhibited faster electrophoretic migration.

FIG. 5D is a bar graph showing that Wnt3a produced from Tiki2-expressing L cells exhibited minimal activity in activation of TOPFLASH reporter expression.

FIG. 5E is a gel showing that Wnt3a produced from Tiki2-expressing L cells induced minimal LRP6 or Dvl2 phosphorylation, or beta-catenin stabilization. L cells were treated with control CM or increasing amount of Wnt3a CM for 2 hrs, and the WCL and the input Wnt3a CM were analyzed by Western blotting. LRP6 protein levels were not altered upon Wnt3a treatment.

FIG. 5F is a gel showing that Wnt3a secreted from Tiki2-expressing cells exhibited minimal binding to either Fz8 or LRP6. Wnt3a CM was incubated with LRP6N-IgG or mFz8CRD-IgG. The protein complexes were precipitated by protein G agarose and subjected for Western blotting.

FIG. 6A is a gel showing that Tiki inactivates Wnt3a by regulating Wnt3a acylation/palmitoylation. A Triton X-114 phase separation experiment is shown in 6A. Wnt3a from control cells mainly partitioned in the detergent phase, but Wnt3a produced from Tiki2-expressing cells partitioned exclusively in the aqueous phase. T, total; Aq, aqueous; De, detergent.

FIG. 6B is a gel showing that Wnt3a secreted from Tiki1 or Tiki2 but not GFP-expressing HEK293T cells partitioned in the aqueous phase.

FIG. 6C is a gel showing the results of Acyl-biotinyl exchange (ABE) analysis of control Wnt3a or Wnt3a produced from Tiki2-expressing cells. Biotinylation of Wnt3a secreted from Tiki2-expressing cells was much reduced compared to that of Wnt3a from control cells.

FIG. 6D is a gel showing that Wnt3a secreted from Tiki2N-, but not Krm2N- or Tiki2NΔ-expressing HEK293T cells partitioned exclusively in the aqueous phase.

FIG. 6E is a gel showing that Tiki2, but not the control GFP, reduces Wnt3a acylation in HEK293T cells (Top panel). The total amount of Wnt3a is constant (bottom panel).

FIG. 6F is bar graph showing quantification of the data in FIG. 6E, demonstrating that Tiki2 reduces Wnt3a acylation by about 31%.

FIG. 6G is a gel showing that recombinant WNT3A was incubated with Krm2N or Tiki2N purified from WCL (top) or from CM (bottom) and was subjected to Triton X-114 phase separation.

FIG. 6H is a gel showing that Recombinant WNT3A was incubated with Krm2N or Tiki2N or Tiki2NΔpurified from WCL and was subjected to Triton X-114 phase separation.

FIG. 6I is a gel showing the results of ABE analysis of WNT3A after in vitro incubation with Krm2N or Tiki2N. Biotinylation of WNT3A was much reduced after incubation with Tiki2N compared to that of WNT3A incubated with Krm2N.

FIG. 6J is a gel showing the results of silver staining of purified proteins used for in vitro assays for 6E to 6G.

FIGS. 7A-C are each schematics with accompanying bar graphs showing that Tiki1 inhibits Wnt/β-catenin signaling in both Wnt-producing and Wnt-responding cells. 7A, Tiki1 acts in Xwnt8-producing cells. Xwnt8 mRNA (20 pg) was co-injected with Tiki1 or LDLR mRNA (100 pg) into a single blastomere at the 8-cell stage, while S01234-Luciferase (140 pg) plus TK-Renilla (1.25 pg) reporter DNAs were injected into a neighboring blastomere. 7B, Tiki1 acts in Xwnt8-responding cells. S01234-Luciferase and TK-Renilla reporter DNAs were co-injected with Tiki1 or LDLR mRNA in a single blastomere, while Xwnt8 mRNA was injected into a neighboring blastomere. Luciferase readings were normalized to Renilla. 7C, Tiki1 reduces nuclear beta-catenin levels in Xwnt8-responding cells. Tiki1 or LDLR mRNA (200 pg) together with fluorescein dextran (FLD) were injected into one blastomere at the 8-cell stage, while Xwnt8 mRNA (20 pg) plus RFP mRNA (800 pg) were injected into a neighboring blastomere. Stage 9 animal cap cells were subjected to immunofluorescence analysis with anti-beta-catenin antibodies. Only cells within the distance of five cell bodies from Xwnt8-expressing cells were counted in any given field. No nuclear β-catenin-positive cells were found in areas free of Xwnt8-expressing cells (not shown). Statistical data were derived from three independent experiments. **p<0.001.

FIG. 7D is an immunoblot showing the results of experiments in HeLa cells expressing HA-Tiki1 or HA-Tiki2 and labeled by a cell non-permeable biotinylation reagent. Cell surface (CS) proteins were precipitated with streptavidin agarose beads and subjected to immunoblotting analysis using an anti-HA antibody. Input WCL were also analyzed by the HA antibody. HA-Tiki proteins exhibited two forms. The slower migrating form was enriched on the cell surface, and might be more extensively glycosylated.

FIG. 7E is an immunoblot showing that HA-Tiki1N and HA-Tiki2N were secreted in CM. Secreted HA-Tiki proteins, which might be more extensively glycosylated, migrated slower in SDS-PAGE than those in WCL.

FIG. 8 is a set of three images showing the Tiki2 high bone mass phenotype. Femur cross sections of 16 week old male +/+, Tiki2 +/− and Tiki2−/− in representative median animals (image generated from μCT composite).

FIG. 9 is a schematic and a set of five bar graphs showing quantitation of Tiki2 high bone mass phenotype by μCT of trabecualar bone. A 2 mm region of interest in the distal femur was analyzed for a cohort of 16 week old male mice. +/+n=7, +/−n=10, −/−n=6. Significant differences compared to +/+ levels are noted with asterisks: p<0.05 *, p<0.01 **.

FIGS. 10 and 11 show the protein and nucleic acid sequences of human Tiki1 and Tiki2, respectively.

FIG. 12 shows the sequences of the human Tiki1N and Tiki2N truncated forms.

### DETAILED DESCRIPTION

The present invention is based, at least in part, on the identification of a new family of Wnt antagonists, referred to as the Tiki family. Tiki genes encode single-span transmembrane proteins that specifically antagonize Wnt signaling. Unlike Sclerostin or DKK1 that binds to and inhibits LRP5/6, or SFRP1 that binds to and inhibits Wnt/Fz (15), Tiki proteins appear to modify and inactivate Wnt ligands, rendering them incapable of binding to either LRP5/6 or Fz. Tiki proteins likely act as enzymes that alter lipid modification required for Wnt activity. The findings described herein suggest a new paradigm for regulating Wnt signaling through lipid modification of secreted ligands. We have generated Tiki2−/− mutant mice, which surprisingly are viable due possibly to compensation by other Wnt antagonists. Interesting Tiki2 is expressed in osteoblasts postnatally and the Tiki2−/− mice exhibit high bone mass, suggesting that Tiki2, like Sclerostin, DKK1, and SFRP1, may be an important negative regulator of bone homeostasis. Because Wnt/LRP regulation of bone mass is highly conserved between mice and men, human Tiki2 therefore represents a new therapeutic target for treating osteoporosis and bone fractures, as well as cellular proliferative disorders.

WNT Signaling

Signaling by the Wnt family of secreted lipoproteins through LDL receptor-related proteins 5 and 6 (LRP5 and LRP6) has emerged as a key pathway that controls bone mass (Baron et al., Curr Osteoporos Rep. 2007. 5:73-80). Human familial osteoporosis and high bone mass (HBM) diseases have been linked to loss-of-function and ‘gain-of-function’ mutations in LRP5, respectively (Gong et al., Cell. 2001. 107:513-23; Boyden et al., N Engl J. Med. 2002. 346:1513-21; Little et al., Am J Hum Genet. 2002. 70:11-9), and a hypomorphic LRP6 mutation is also linked to osteoporosis (Mani et al., Science. 2007. 315:1278-82). Genetic manipulations of Lrp5 and Lrp6 in mice fully recapitulate human disease conditions (Holmen et al. J Bone Miner Res. 2004. 19:2033-40; Kato et al., J. Cell Biol. 2002. 157:303-14). These studies indicate that LRP5 and possibly LRP6 are major players in stimulating bone growth in mice and human (Baron et al., Curr Osteoporos Rep. 2007. 5:73-80).

LRP5 and LRP6 are coreceptors for the Frizzled (Fz) family of Wnt receptors (He et al., Development. 2004. 131:1663-77). Wnt engagement of the Fz-LRP receptor complex activates a signaling pathway through stabilization of the transcription co-activator β-catenin, leading to Wnt-responsive gene expression. Although one study suggested that LRP5 regulates intestinal synthesis of serotonin, which in turn regulates bone mass (Yadav et al., Cell. 2008. 135:825-37), the evidence that LRP5 mediates Wnt signaling in the bone is vast and substantial. Several secreted Wnt antagonists, such as Sclerostin, DICKKOPF1 (DKK1), and secreted Frizzled-related protein 1 (SFRP1), have been shown to express in the bone and negatively regulate bone mass in human and/or mice. In fact human patients deficient for Sclerostin exhibit sclerosteosis (Baron et al. Endocrinology. 2007. 148:2635-43) or Van Buchem disease (Balemans et al., J Med Genet. 2002. 39:91-7), both of which are characterized by excessive bone growth, and genetic deletion of the Sclerostin gene and antibody-blockage of Sclerostin in experimental animals lead to increased bone mass (Hoeppner et al., 2009. 13:485-96; Li et al. J Bone Miner Res. 2009. 24:578-88). These studies not only demonstrate a pivotal ‘yin-yang’ role of Wnt antagonists and LRP5/LRP6 in bone tissue homeostasis, but also suggest that Wnt antagonists are potential therapeutic targets for treating osteoporosis and stimulating bone regeneration. Because Wnt/LRP signaling primarily regulates osteoblast proliferation and differentiation and thus bone tissue generation, manipulating Wnt/LRP signaling represents anabolic therapeutics; which is sorely lacking in the current strategies for treating osteoporosis through the prevention of bone resorption.

Tiki Nucleic Acid Molecules

In one aspect, the invention provides isolated nucleic acid molecules that encode a Tiki1/2 polypeptide described herein, e.g., a full length Tiki1/2 protein. Also included is a nucleic acid fragment suitable for use as a hybridization probe, which can be used, e.g., to identify a nucleic acid molecule encoding a polypeptide of the invention, Tiki1/2 mRNA, and fragments suitable for use as primers, e.g., PCR primers for the amplification or mutation of Tiki1/2 nucleic acid molecules.

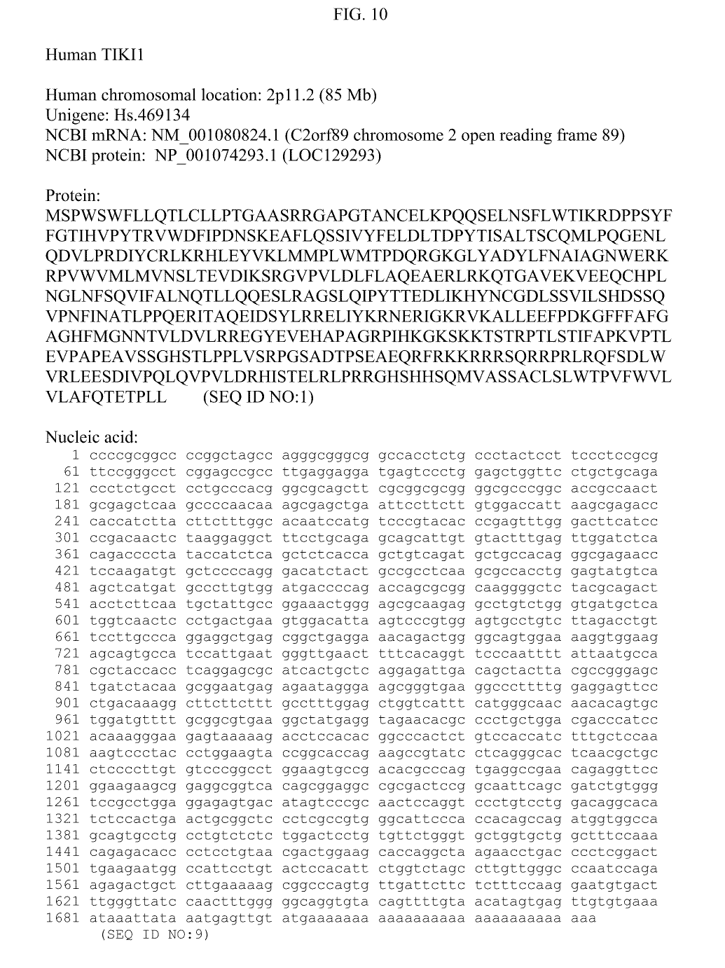

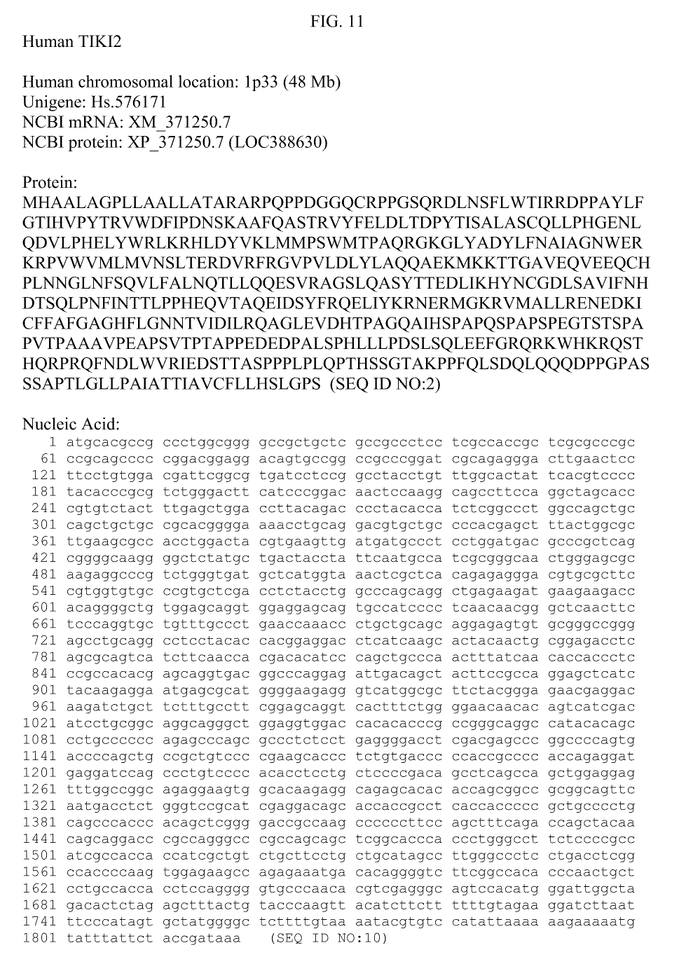

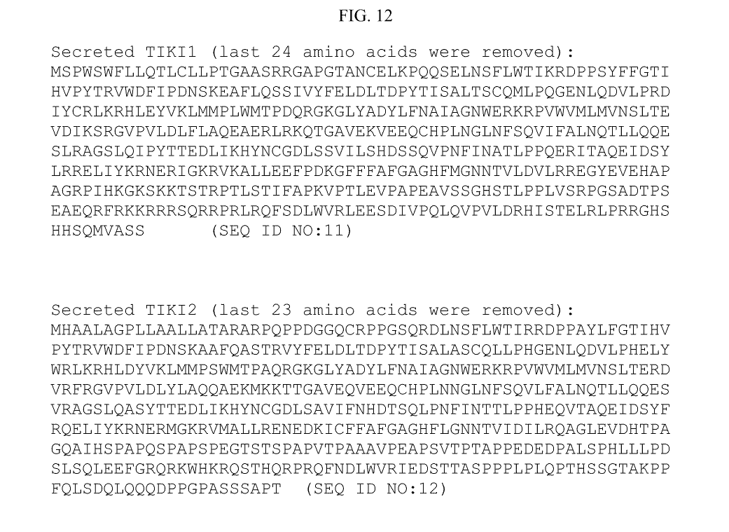

In some embodiments, an isolated nucleic acid molecule of the invention includes a nucleotide sequence shown in FIG. 10 or 11. In some embodiments, the nucleic acid molecule includes sequences encoding the human Tiki1/2 protein (i.e., “the coding region”), as well as 5′ untranslated sequences. In some embodiments, the nucleic acid molecule includes sequences encoding a truncated version of a TIKI1/2 protein, e.g., a version that lacks the C-terminal transmembrane domain, e.g., as shown in FIG. 12. Alternatively, the nucleic acid molecule can include only the coding region of a sequence shown in FIG. 10 or 11 and, e.g., no flanking sequences which normally accompany the subject sequence.

In another embodiment, a Tiki1/2 nucleic acid molecule includes a nucleic acid molecule which is a complement of a sequence described herein. In other embodiments, a Tiki1/2 nucleic acid molecule is sufficiently complementary to a nucleotide sequence described herein that it can hybridize to the nucleotide sequence under stringent conditions, thereby forming a stable duplex.

In one embodiment, a Tiki1/2 isolated nucleic acid molecule includes a nucleotide sequence which is at least about 85% or more homologous to the entire length of a nucleotide sequence shown in FIG. 10 or 11. In some embodiments, the nucleotide sequence is at least about 90%, 91%, 92%, 93%, 94%, 95%, 96%, 97%, 98%, 99% or 100% to a nucleotide sequence shown in FIG. 10 or 11.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

For purposes of the present invention, the comparison of sequences and determination of percent identity between two sequences can be accomplished using a Blossum 62 scoring matrix with a gap penalty of 12, a gap extend penalty of 4, and a frameshift gap penalty of 5.

Tiki1/2 Nucleic Acid Fragments

A nucleic acid molecule of the invention can include only a portion of a nucleic acid sequence shown in FIG. 10 or 11. For example, such a nucleic acid molecule can include a fragment which can be used as a probe or primer, e.g., directed to a sequence of a splice site not present in the full-length Tiki1/2.

Thus, Tiki1/2 probes and primers are provided. Typically a probe/primer is an isolated or purified oligonucleotide. The oligonucleotide typically includes a region of nucleotide sequence that hybridizes under stringent conditions to at least about 15 consecutive nucleotides of a sense or antisense sequence a nucleic acid sequence shown in FIG. 10 or 11. In some embodiments, the oligonucleotide comprises about 20, 25, 30, 35, 40, 45, 50, 55, 60, 65, or 75 consecutive nucleotides of a nucleic acid sequence shown in FIG. 10 or 11.

In some embodiments, the nucleic acid is a probe which is at least 10, and less than 200 (typically less than about 100 or 50) base pairs in length. It should be identical, or differ by 1, or less than 1 in 5 or 10 bases, from a sequence disclosed herein. If alignment is needed for this comparison the sequences should be aligned for maximum homology. “Looped” out sequences from deletions or insertions, or mismatches, are considered differences.

In another embodiment a set of primers is provided, e.g., primers suitable for use in a PCR, which can be used to amplify a selected region of a Tiki1/2 sequence, e.g., to detect the presence of a specific variant. The primers should be at least 20 base pairs in length and less than about 100 base pairs in length. The primers should be identical, or differ by one base from a sequence disclosed herein or from a naturally occurring variant.

A nucleic acid fragment can encode an epitope-bearing region of a polypeptide described herein, e.g., an antigenic epitope specific to the variant.

A nucleic acid fragment encoding a “biologically active portion of a Tiki1/2 polypeptide” can be prepared by isolating a portion of a nucleic acid sequence shown in FIG. 10 or 11, which encodes a polypeptide having a Tiki1/2 biological activity (e.g., the biological activities of the Tiki1/2 proteins are described herein), expressing the encoded portion of the Tiki1/2 protein (e.g., by recombinant expression in vitro) and assessing the activity of the encoded portion of the Tiki1/2 protein. A nucleic acid fragment encoding a biologically active portion of a Tiki1/2 polypeptide, may comprise a nucleotide sequence which is greater than 300 or more nucleotides in length.

In preferred embodiments, a nucleic acid includes a nucleotide sequence which is about 300, 400, 500, 600, 700, 800, 900, 1000, 1100, 1200, 1300 or more nucleotides in length and hybridizes under stringent hybridization conditions to a nucleic acid sequence shown in FIG. 10 or 11.

Tiki1/2 Nucleic Acid Variants

The invention further encompasses nucleic acid molecules that differ from a nucleic acid sequence shown in FIG. 10 or 11. Such differences can be due to degeneracy of the genetic code (and result in a nucleic acid which encodes the same Tiki1/2 proteins as those encoded by the nucleotide sequence disclosed herein. In another embodiment, an isolated nucleic acid molecule of the invention has a nucleotide sequence encoding a protein having an amino acid sequence which differs, by at least 1, but less than 5, 10, 20, 50, or 100 amino acid residues from that shown in FIG. 10 or 11. If alignment is needed for this comparison the sequences should be aligned for maximum homology. “Looped” out sequences from deletions or insertions, or mismatches, are considered differences.

Nucleic acids of the inventor can be chosen for having codons, which are preferred, or non preferred, for a particular expression system. E.g., the nucleic acid can be one in which at least one codon, at preferably at least 10%, or 20% of the codons has been altered such that the sequence is optimized for expression in E. coli, yeast, human, insect, or CHO cells.

Nucleic acid variants can be naturally occurring, such as allelic variants (same locus), homologs (different locus), and orthologs (different organism) or can be non naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

In a preferred embodiment, the nucleic acid differs from a nucleic acid sequence shown in FIG. 10 or 11, e.g., as follows: by at least one but less than 10, 20, 30, or 40 nucleotides; at least one but less than 1%, 5%, 10% or 20% of the nucleotides in the subject nucleic acid. If necessary for this analysis the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a polypeptide that is 50%, at least about 55%, typically at least about 70-75%, more typically at least about 80-85%, and most typically at least about 90-95% or more identical to the nucleotide sequence shown in FIG. 10 or 11 or a fragment of this sequence. Such nucleic acid molecules can readily be identified as being able to hybridize under stringent conditions, to the nucleotide sequence shown in FIG. 10 or 11 or a fragment of the sequence, or to the complement thereof. Nucleic acid molecules corresponding to orthologs, homologs, and allelic variants of the Tiki1/2 cDNAs of the invention can further be isolated by mapping to the same chromosome or locus as the Tiki1/2 gene.

Preferred variants include those that are correlated with an activity described herein.

Allelic variants of Tiki1/2, e.g., human Tiki1/2, include both functional and non-functional proteins. Functional allelic variants are naturally occurring amino acid sequence variants of the Tiki1/2 protein within a population that maintain the ability to modulate Wnt signalling. Functional allelic variants will typically contain only conservative substitution of one or more amino acids of a sequence shown in FIG. 10 or 11 or 12, or substitution, deletion or insertion of non-critical residues in non-critical regions of the protein. Non-functional allelic variants are naturally-occurring amino acid sequence variants of the Tiki1/2, e.g., human Tiki1/2, protein within a population that do not have the ability to modulate Wnt signalling. Non-functional allelic variants will typically contain a non-conservative substitution, a deletion, or insertion, or premature truncation of the amino acid sequence shown in FIG. 10 or 11, or a substitution, insertion, or deletion in critical residues or critical regions of the protein.

Moreover, nucleic acid molecules encoding other Tiki1/2 family members and, thus, which have a nucleotide sequence that differs from the Tiki1/2 sequences shown in FIG. 10 or 11 are intended to be within the scope of the invention.

Inhibitory Nucleic Acids

Inhibitory nucleic acids, e.g., siRNA, antisense, ribozymes, or aptamers, directed against Tiki1/2, can also be used.

RNA Interference

RNA interference (RNAi) is a process that induces the sequence-specific regulation of gene expression in animal and plant cells and in bacteria (Aravin and Tuschl, FEBS Lett. 26:5830-5840 (2005); Herbert et al., Curr. Opin. Biotech. 19:500-505 (2008); Hutvagner and Zamore, Curr. Opin. Genet. Dev.:12, 225-232 (2002); Sharp, Genes Dev., 15:485-490 (2001); Valencia-Sanchez et al. Genes Dev. 20:515-524 (2006)). In mammalian cells, RNAi can be triggered by 21-nucleotide (nt) duplexes of small interfering RNA (siRNA) (Chiu et al., Mol. Cell. 10:549-561 (2002); Elbashir et al., Nature 411:494-498 (2001)), by microRNA (miRNA), functional small-hairpin RNA (shRNA), or other double stranded RNAs (dsRNAs) which are expressed in vivo using DNA templates with RNA polymerase II or III promoters (Zeng et al., Mol. Cell. 9:1327-1333 (2002); Paddison et al., Genes Dev. 16:948-958 (2002); Denti, et al., Mol. Ther. 10:191-199 (2004); Lee et al., Nature Biotechnol. 20:500-505 (2002); Paul et al., Nature Biotechnol. 20:505-508 (2002); Rossi, Human Gene Ther. 19:313-317 (2008); Tuschl, T., Nature Biotechnol. 20:440-448 (2002); Yu et al., Proc. Natl. Acad. Sci. USA 99(9):6047-6052 (2002); McManus et al., RNA 8:842-850 (2002); Scherer et al., Nucleic Acids Res. 35:2620-2628 (2007); Sui et al., Proc. Natl. Acad. Sci. USA 99(6):5515-5520 (2002).)

siRNA Molecules

In some embodiments, the methods described herein can use dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is complementary to the first strand. The dsRNA molecules can be chemically synthesized, or can transcribed be in vitro or in vivo, e.g., shRNA, from a DNA template. The dsRNA molecules can be designed using any method known in the art. Negative control siRNAs should not have significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

The methods described herein can use both siRNA and modified siRNA derivatives, e.g., siRNAs modified to alter a property such as the specificity and/or pharmacokinetics of the composition, for example, to increase half-life in the body, e.g., crosslinked siRNAs. Thus, the methods can include administering siRNA derivatives that include siRNA having two complementary strands of nucleic acid, such that the two strands are crosslinked. The oligonucleotide modifications include, but are not limited to, 2′-O-methyl, 2′-fluoro, 2′-O-methyoxyethyl and phosphorothiate, boranophosphate, 4′-thioribose. (Wilson and Keefe, Curr. Opin. Chem. Biol. 10:607-614 (2006); Prakash et al., J. Med. Chem. 48:4247-4253 (2005); Soutschek et al., Nature 432:173-178 (2004))

In some embodiments, the siRNA derivative has at its 3′ terminus a biotin molecule (e.g., a photocleavable biotin), a peptide (e.g., a Tat peptide), a nanoparticle, a peptidomimetic, organic compounds (e.g., a dye such as a fluorescent dye), or dendrimer. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The inhibitory nucleic acid compositions can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished by methods known in the art, e.g., using the methods of Lambert et al., Drug Deliv. Rev.: 47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles). The inhibitory nucleic acid molecules can also be labeled using any method known in the art; for instance, the nucleic acid compositions can be labeled with a fluorophore, e.g., Cy3, fluorescein, or rhodamine. The labeling can be carried out using a kit, e.g., the SILENCER™ siRNA labeling kit (Ambion). Additionally, the siRNA can be radiolabeled, e.g., using ³H, ³²P, or other appropriate isotope.

siRNA Delivery

Direct delivery of siRNA in saline or other excipients can silence target genes in tissues, such as the eye, lung, and central nervous system (Bitko et al., Nat. Med. 11:50-55 (2005); Shen et al., Gene Ther. 13:225-234 (2006); Thakker, et al., Proc. Natl. Acad. Sci. U.S.A. (2004)). In adult mice, efficient delivery of siRNA can be accomplished by “high-pressure” delivery technique, a rapid injection (within 5 seconds) of a large volume of siRNA containing solution into animal via the tail vein (Liu (1999), supra; McCaffrey (2002), supra; Lewis, Nature Genetics 32:107-108 (2002)).

Liposomes and nanoparticles can also be used to deliver siRNA into animals. Delivery methods using liposomes, e.g. stable nucleic acid-lipid particles (SNALPs), dioleoyl phosphatidylcholine (DOPC)-based delivery system, as well as lipoplexes, e.g. Lipofectamine 2000, TransIT-TKO, have been shown to effectively repress target mRNA (de Fougerolles, Human Gene Ther. 19:125-132 (2008); Landen et al., Cancer Res. 65:6910-6918 (2005); Luo et al., Mol. Pain. 1:29 (2005); Zimmermann et al., Nature 441:111-114 (2006)). Conjugating siRNA to peptides, RNA aptamers, antibodies, or polymers, e.g. dynamic polyconjugates, cyclodextrin-based nanoparticles, atelocollagen, and chitosan, can improve siRNA stability and/or uptake. (Howard et al., Mol. Ther. 14:476-484 (2006); Hu-Lieskovan et al., Cancer Res. 65:8984-8992 (2005); Kumar, et al., Nature 448:39-43; McNamara et al., Nat. Biotechnol. 24:1005-1015 (2007); Rozema et al., Proc. Natl. Acad. Sci. U.S.A. 104:12982-12987 (2007); Song et al., Nat. Biotechnol. 23:709-717 (2005); Soutschek (2004), supra; Wolfrum et al., Nat. Biotechnol. 25:1149-1157 (2007))

Viral-mediated delivery mechanisms can also be used to induce specific silencing of targeted genes through expression of siRNA, for example, by generating recombinant adenoviruses harboring siRNA under RNA Pol II promoter transcription control (Xia et al. (2002), supra). Infection of HeLa cells by these recombinant adenoviruses allows for diminished endogenous target gene expression. Injection of the recombinant adenovirus vectors into transgenic mice expressing the target genes of the siRNA results in in vivo reduction of target gene expression. Id. In an animal model, whole-embryo electroporation can efficiently deliver synthetic siRNA into post-implantation mouse embryos (Calegari et al., Proc. Natl. Acad. Sci. USA 99(22):14236-40 (2002)).

Stable siRNA Expression

Synthetic siRNAs can be delivered into cells, e.g., by direct delivery, cationic liposome transfection, and electroporation. However, these exogenous siRNA typically only show short term persistence of the silencing effect (4˜5 days). Several strategies for expressing siRNA duplexes within cells from recombinant DNA constructs allow longer-term target gene suppression in cells, including mammalian Pol II and III promoter systems (e.g., H1, U1, or U6/snRNA promoter systems (Denti et al. (2004), supra; Tuschl (2002), supra); capable of expressing functional double-stranded siRNAs (Bagella et al., J. Cell. Physiol. 177:206-213 (1998); Lee et al. (2002), supra; Miyagishi et al. (2002), supra; Paul et al. (2002), supra; Scherer et al. (2007), supra; Yu et al. (2002), supra; Sui et al. (2002), supra).

Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al. (1998), supra; Lee et al. (2002), supra; Miyagishi et al. (2002), supra; Paul et al. (2002), supra; Yu et al. (2002), supra; Sui et al. (2002) supra). Constructs containing siRNA sequence under the control of T7 promoter also make functional siRNAs when cotransfected into the cells with a vector expression T7 RNA polymerase (Jacque (2002), supra).

In another embodiment, siRNAs can be expressed in a miRNA backbone which can be transcribed by either RNA Pol II or III. MicroRNAs are endogenous noncoding RNAs of approximately 22 nucleotides in animals and plants that can post-transcriptionally regulate gene expression (Bartel, Cell 116:281-297 (2004); Valencia-Sanchez et al., Genes & Dev. 20:515-524 (2006)) One common feature of miRNAs is that they are excised from an approximately 70 nucleotide precursor RNA stem loop by Dicer, an RNase III enzyme, or a homolog thereof. By substituting the stem sequences of the miRNA precursor with the sequence complementary to the target mRNA, a vector construct can be designed to produce siRNAs to initiate RNAi against specific mRNA targets in mammalian cells. When expressed by DNA vectors containing polymerase II or III promoters, miRNA designed hairpins can silence gene expression (McManus (2002), supra; Zeng (2002), supra).

Uses of Engineered RNA Precursors to Induce RNAi

Engineered RNA precursors, introduced into cells or whole organisms as described herein, will lead to the production of a desired siRNA molecule. Such an siRNA molecule will then associate with endogenous protein components of the RNAi pathway to bind to and target a specific mRNA sequence for cleavage, destabilization, and/or translation inhibition destruction. In this fashion, the mRNA to be targeted by the siRNA generated from the engineered RNA precursor will be depleted from the cell or organism, leading to a decrease in the concentration of the protein encoded by that mRNA in the cell or organism.

Antisense

An “antisense” nucleic acid can include a nucleotide sequence that is complementary to a “sense” nucleic acid encoding a protein, e.g., complementary to the coding strand of a double-stranded cDNA molecule or complementary to a target mRNA sequence. The antisense nucleic acid can be complementary to an entire coding strand of a target sequence, or to only a portion thereof (for example, the coding region of a target gene). In another embodiment, the antisense nucleic acid molecule is antisense to a “noncoding region” of the coding strand of a nucleotide sequence encoding the selected target gene (e.g., the 5′ and 3′ untranslated regions).

An antisense nucleic acid can be designed such that it is complementary to the entire coding region of a target mRNA but can also be an oligonucleotide that is antisense to only a portion of the coding or noncoding region of the target mRNA. For example, the antisense oligonucleotide can be complementary to the region surrounding the translation start site of the target mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

Based upon the sequences disclosed herein, one of skill in the art can easily choose and synthesize any of a number of appropriate antisense molecules for use in accordance with the present invention. For example, a “gene walk” comprising a series of oligonucleotides of 15-30 nucleotides spanning the length of a target nucleic acid can be prepared, followed by testing for inhibition of target gene expression. Optionally, gaps of 5-10 nucleotides can be left between the oligonucleotides to reduce the number of oligonucleotides synthesized and tested.

The antisense nucleic acid molecules of the invention are typically administered to a subject (e.g., by direct injection at a tissue site), or generated in situ such that they hybridize with or bind to cellular mRNA and/or genomic DNA encoding a target protein to thereby inhibit expression of the protein, e.g., by inhibiting transcription, splicing, and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter can be used.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al., Nucleic Acids. Res. 15:6625-6641 (1987)). The antisense nucleic acid molecule can also comprise a 2′-O-methylribonucleotide (Inoue et al. Nucleic Acids Res. 15:6131-6148 (1987)), 2′-O-methoxyethylribonucleotide, locked nucleic acid, ethylene-bridged nucleic acid, oxetane-modified ribose, peptide nucleic acid, or a chimeric RNA-DNA analogue (Inoue et al. FEBS Lett., 215:327-330 (1987)).

In some embodiments, the antisense nucleic acid is a morpholino oligonucleotide (see, e.g., Heasman, Dev. Biol. 243:209-14 (2002); Iversen, Curr. Opin. Mol. Ther. 3:235-8 (2001); Summerton, Biochim. Biophys. Acta. 1489:141-58 (1999).

Target gene expression can be inhibited by targeting nucleotide sequences complementary to a regulatory region, e.g., promoters and/or enhancers) to form triple helical structures that prevent transcription of the target gene in target cells. See generally, Helene, C. Anticancer Drug Des. 6:569-84 (1991); Helene, C. Ann. N.Y. Acad. Sci. 660:27-36 (1992); and Maher, Bioassays 14:807-15 (1992). The potential sequences that can be targeted for triple helix formation can be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

Ribozymes

Ribozymes are a type of RNA that can be engineered to enzymatically synthesize, or cleave and inactivate, other RNA targets in a specific, sequence-dependent fashion. By cleaving the target RNA, ribozymes inhibit translation, thus preventing the expression of the target gene. Ribozymes can be chemically synthesized in the laboratory and structurally modified to increase their stability and catalytic activity using methods known in the art. Alternatively, ribozyme genes can be introduced into cells through gene-delivery mechanisms known in the art. A ribozyme having specificity for a target-protein encoding nucleic acid can include one or more sequences complementary to the nucleotide sequence of a target cDNA disclosed herein, and a sequence having known catalytic sequence responsible for mRNA cleavage (see U.S. Pat. No. 5,093,246 or Haselhoff and Gerlach Nature 334:585-591 (1988)). For example, a derivative of a Tetrahymena L-19 IVS RNA can be constructed in which the nucleotide sequence of the active site is complementary to the nucleotide sequence to be cleaved in a target mRNA. See, e.g., Cech et al. U.S. Pat. No. 4,987,071; and Cech et al. U.S. Pat. No. 5,116,742. Alternatively, a target mRNA can be used to select a catalytic RNA having a specific ribonuclease activity from a pool of RNA molecules. See, e.g., Bartel and Szostak, Science 261:1411-1418 (1993).

Isolated Tiki1/2 Polypeptides

In another aspect, the invention features isolated Tiki1/2 proteins, or fragments, e.g., a biologically active portion, for use as ligand-binding fragments or as immunogens or antigens to raise or test (or more generally to bind) anti-Tiki1/2 antibodies. Tiki1/2 protein can be isolated from cells or tissue sources using standard protein purification techniques. Tiki1/2 protein or fragments thereof can be produced by recombinant DNA techniques or synthesized chemically. In some embodiments, the fragment is a truncated version of a TIKI1/2 protein, e.g., a version that lacks the C-terminal transmembrane domain, e.g., as shown in FIG. 12.

Polypeptides of the invention include those which arise as a result of the existence of multiple genes, alternative transcription events, alternative RNA splicing events, and alternative translational and post-translational events. The polypeptide can be expressed in systems, e.g., cultured cells, which result in substantially the same post-translational modifications present when expressed the polypeptide is expressed in a native cell, or in systems which result in the alteration or omission of post-translational modifications, e.g., glycosylation or cleavage, present when expressed in a native cell.

In a preferred embodiment the Tiki1/2 protein, or fragment thereof, differs from the corresponding sequence shown in FIG. 10 or 11 or 12. In one embodiment it differs by at least one but by less than 15, 10 or 5 amino acid residues. In another it differs from the corresponding sequence shown in FIG. 10 or 11 or 12 by at least one residue but less than 20%, 15%, 10% or 5% of the residues in it differ from the corresponding sequence shown in FIG. 10 or 11 or 12. (If this comparison requires alignment the sequences should be aligned for maximum homology. “Looped” out sequences from deletions or insertions, or mismatches, are considered differences.) The differences are, preferably, differences or changes at a non essential residue or a conservative substitution. In some embodiments the differences are not in the Tiki domain. In another embodiment one or more differences are in the Tiki domain.

Other embodiments include a protein that contain one or more changes in amino acid sequence, e.g., a change in an amino acid residue which is not essential for activity. Such Tiki1/2 proteins differ in amino acid sequence from those shown in FIG. 10 or 11 or 12 yet retain biological activity.

In one embodiment, the protein includes an amino acid sequence at least about 80%, 85%, 90%, 95%, 98% or more homologous to that shown in FIG. 10 or 11 or 12.

A Tiki1/2 protein or fragment is also provided which varies from the sequence shown in FIG. 10 or 11 or 12 by at least one but by less than 15, 10 or 5 amino acid residues in the protein or fragment but which does not differ from a sequence shown in FIG. 10 or 11 or 12 in a Tiki/GumN region. (If this comparison requires alignment the sequences should be aligned for maximum homology. “Looped” out sequences from deletions or insertions, or mismatches, are considered differences.) In some embodiments the difference is at a non essential residue or is a conservative substitution, while in others the difference is at an essential residue or is a non conservative substitution.

In one embodiment, a biologically active portion of a Tiki1/2 protein includes a Tiki domain. Moreover, other biologically active portions, in which other regions of the protein are deleted, can be prepared by recombinant techniques and evaluated for one or more of the functional activities of a native Tiki1/2 protein.

In a preferred embodiment, the Tiki1/2 protein has an amino acid sequence shown in FIG. 10 or 11 or 12. In other embodiments, the Tiki1/2 protein is substantially identical to that shown in FIG. 10 or 11 or 12. In yet another embodiment, the Tiki1/2 protein is substantially identical to shown in that FIG. 10 or 11 or 12 and retains the functional activity of the protein shown in FIG. 10 or 11 or 12, as described in detail herein.

Tiki1/2 Chimeric or Fusion Proteins

In another aspect, the invention provides Tiki1/2 chimeric or fusion proteins. As used herein, a Tiki1/2 “chimeric protein” or “fusion protein” includes a Tiki1/2 polypeptide linked to a non-Tiki1/2 polypeptide. A “non-Tiki1/2 polypeptide” refers to a polypeptide having an amino acid sequence corresponding to a protein which is not substantially homologous to the Tiki1/2 protein, e.g., a protein which is different from the Tiki1/2 protein and which is derived from the same or a different organism. The Tiki1/2 polypeptide of the fusion protein can correspond to all or a portion e.g., a fragment described herein of a Tiki1/2 amino acid sequence (e.g., as shown in FIG. 12). In a preferred embodiment, a Tiki1/2 fusion protein includes at least one (or two) biologically active portion of a Tiki1/2 protein. The non-Tiki1/2 polypeptide can be fused to the N-terminus or C-terminus of the Tiki1/2 polypeptide.

The fusion protein can include a moiety which has a high affinity for a ligand. For example, the fusion protein can be a GST-Tiki1/2 fusion protein in which the Tiki1/2 sequences are fused to the C-terminus of the GST sequences. Such fusion proteins can facilitate the purification of recombinant Tiki1/2. Alternatively, the fusion protein can be a Tiki1/2 protein containing a heterologous signal sequence at its N-terminus. In certain host cells (e.g., mammalian host cells), expression and/or secretion of Tiki1/2 can be increased through use of a heterologous signal sequence.

Fusion proteins can include all or a part of a serum protein, e.g., an IgG constant region, or human serum albumin.

In some embodiments, the fusion protein includes a cell-penetrating peptide sequence that facilitates delivery of Tiki1/2 to the intracellular space, e.g., HIV-derived TAT peptide, penetratins, transportans, or hCT derived cell-penetrating peptides, see, e.g., Caron et al., (2001) Mol. Ther. 3(3):310-8; Langel, Cell-Penetrating Peptides: Processes and Applications (CRC Press, Boca Raton Fla. 2002); El-Andaloussi et al., (2005) Curr Pharm Des. 11(28):3597-611; and Deshayes et al., (2005) Cell Mol Life Sci. 62(16): 1839-49.

The Tiki1/2 fusion proteins of the invention can be incorporated into pharmaceutical compositions and administered to a subject in vivo. The Tiki1/2 fusion proteins can be used to affect the bioavailability of a Tiki1/2 substrate. Tiki1/2 fusion proteins may be useful therapeutically for the treatment of disorders caused by, for example, (i) aberrant modification or mutation of a gene encoding a Tiki1/2 protein; (ii) mis-regulation of the Tiki1/2 gene; and (iii) aberrant post-translational modification of a Tiki1/2 protein.

Moreover, the Tiki1/2-fusion proteins of the invention can be used as immunogens to produce anti-Tiki1/2 antibodies in a subject, to purify Tiki1/2 ligands and in screening assays to identify molecules which inhibit the interaction of Tiki1/2 with a Tiki1/2 substrate.

Expression vectors are commercially available that already encode a fusion moiety (e.g., a GST polypeptide). A Tiki1/2-encoding nucleic acid can be cloned into such an expression vector such that the fusion moiety is linked in-frame to the Tiki1/2 protein.

Variants of Tiki1/2 Proteins

In another aspect, the invention also features a variant of a Tiki1/2 polypeptide, e.g., which functions as an agonist (mimetics) or as an antagonist. Variants of the Tiki1/2 proteins can be generated by mutagenesis, e.g., discrete point mutation, the insertion or deletion of sequences or the truncation of a Tiki1/2 protein. An agonist of the Tiki1/2 proteins can retain substantially the same, or a subset, of the biological activities of the naturally occurring form of a Tiki1/2 protein. An antagonist of a Tiki1/2 protein can inhibit one or more of the activities of the naturally occurring form of the Tiki1/2 protein by, for example, competitively modulating a Tiki1/2-mediated activity of a Tiki1/2 protein. Thus, specific biological effects can be elicited by treatment with a variant of limited function. Preferably, treatment of a subject with a variant having a subset of the biological activities of the naturally occurring form of the protein has fewer side effects in a subject relative to treatment with the naturally occurring form of the Tiki1/2 protein.

Variants of a Tiki1/2 protein can be identified by screening combinatorial libraries of mutants, e.g., truncation mutants, of a Tiki1/2 protein for agonist or antagonist activity.

Libraries of fragments e.g., N terminal, C terminal, or internal fragments, of a Tiki1/2 protein coding sequence can be used to generate a variegated population of fragments for screening and subsequent selection of variants of a Tiki1/2 protein.

Variants in which a cysteine residue is added or deleted or in which a residue which is glycosylated is added or deleted are particularly preferred.

Methods for screening gene products of combinatorial libraries made by point mutations or truncation, and for screening cDNA libraries for gene products having a selected property. Recursive ensemble mutagenesis (REM), a new technique which enhances the frequency of functional mutants in the libraries, can be used in combination with the screening assays to identify Tiki1/2s (Arkin and Yourvan (1992) Proc. Natl. Acad. Sci. USA 89:7811-7815; Delgrave et al. (1993) Protein Engineering 6:327-331).

Cell based assays can be exploited to analyze a variegated Tiki1/2 library. For example, a library of expression vectors can be transfected into a cell line, e.g., a cell line, which ordinarily responds to Tiki1/2 in a substrate-dependent manner. The transfected cells are then contacted with Tiki1/2 and the effect of the expression of the mutant on signaling by the Tiki1/2 substrate can be detected, e.g., by measuring ACTIVITY1. Plasmid DNA can then be recovered from the cells which score for inhibition, or alternatively, potentiation of signaling by the Tiki1/2 substrate, and the individual clones further characterized.

In another aspect, the invention features a method of making a Tiki1/2 polypeptide, e.g., a peptide having a non-wild type activity, e.g., an antagonist, agonist, or super agonist of a naturally occurring Tiki1/2 polypeptide, e.g., a naturally occurring Tiki1/2 polypeptide. The method includes: altering the sequence of a Tiki1/2 polypeptide, e.g., altering the sequence, e.g., by substitution or deletion of one or more residues of a non-conserved region, a domain or residue disclosed herein, and testing the altered polypeptide for the desired activity.

In another aspect, the invention features a method of making a fragment or analog of a Tiki1/2 polypeptide a biological activity of a naturally occurring Tiki1/2 polypeptide. The method includes: altering the sequence, e.g., by substitution or deletion of one or more residues, of a Tiki1/2 polypeptide, e.g., altering the sequence of a non-conserved region, or a domain or residue described herein, and testing the altered polypeptide for the desired activity.

Recombinant Expression Vectors, Host Cells and Genetically Engineered Cells

In another aspect, the invention includes, vectors, preferably expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a Tiki1/2 nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., Tiki1/2 proteins, mutant forms of Tiki1/2 proteins, fusion proteins, and the like).

The recombinant expression vectors of the invention can be designed for expression of Tiki1/2 proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in Tiki1/2 activity assays, (e.g., direct assays or competitive assays described in detail below), or to generate antibodies specific for Tiki1/2 proteins. In a preferred embodiment, a fusion protein expressed in a retroviral expression vector of the present invention can be used to infect bone marrow cells which are subsequently transplanted into irradiated recipients. The pathology of the subject recipient is then examined after sufficient time has passed (e.g., six weeks).

To maximize recombinant protein expression in E. coli is to express the protein in a host bacteria with an impaired capacity to proteolytically cleave the recombinant protein (Gottesman, S., (1990) Gene Expression Technology: Methods in Enzymology 185, Academic Press, San Diego, Calif. 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The Tiki1/2 expression vector can be a yeast expression vector, a vector for expression in insect cells, e.g., a baculovirus expression vector or a vector suitable for expression in mammalian cells.

When used in mammalian cells, the expression vector's control functions are often provided by viral regulatory elements. For example, commonly used promoters are derived from polyoma, Adenovirus 2, cytomegalovirus and Simian Virus 40.

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the alpha-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus. For a discussion of the regulation of gene expression using antisense genes see Weintraub, H. et al., (1986) Antisense RNA as a molecular tool for genetic analysis, Reviews—Trends in Genetics 1:1.

Another aspect the invention provides a host cell which includes a nucleic acid molecule described herein, e.g., a Tiki1/2 nucleic acid molecule within a recombinant expression vector or a Tiki1/2 nucleic acid molecule containing sequences which allow it to homologously recombine into a specific site of the host cell's genome. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a Tiki1/2 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell of the invention can be used to produce (i.e., express) a Tiki1/2 protein. Accordingly, the invention further provides methods for producing a Tiki1/2 protein using the host cells of the invention. In one embodiment, the method includes culturing the host cell of the invention (into which a recombinant expression vector encoding a Tiki1/2 protein has been introduced) in a suitable medium such that a Tiki1/2 protein is produced. In another embodiment, the method further includes isolating a Tiki1/2 protein from the medium or the host cell.

In another aspect, the invention features, a cell or purified preparation of cells which include a Tiki1/2 transgene, or which otherwise misexpress Tiki1/2. The cell preparation can consist of human or non human cells, e.g., rodent cells, e.g., mouse or rat cells, rabbit cells, or pig cells. In preferred embodiments, the cell or cells include a Tiki1/2 transgene, e.g., a heterologous form of a Tiki1/2, e.g., a gene derived from humans (in the case of a non-human cell). The Tiki1/2 transgene can be misexpressed, e.g., overexpressed or underexpressed. In other preferred embodiments, the cell or cells include a gene that misexpresses an endogenous Tiki1/2, e.g., a gene the expression of which is disrupted, e.g., a knockout. Such cells can serve as a model for studying disorders which are related to mutated or mis-expressed Tiki1/2 alleles or for use in drug screening.

In another aspect, the invention features, a human cell, e.g., a hematopoietic stem cell, transformed with nucleic acid which encodes a subject Tiki1/2 polypeptide.

Also provided are cells, preferably human cells, e.g., human hematopoietic or fibroblast cells, in which an endogenous Tiki1/2 is under the control of a regulatory sequence that does not normally control the expression of the endogenous Tiki1/2 gene. The expression characteristics of an endogenous gene within a cell, e.g., a cell line or microorganism, can be modified by inserting a heterologous DNA regulatory element into the genome of the cell such that the inserted regulatory element is operably linked to the endogenous Tiki1/2 gene. For example, an endogenous Tiki1/2 gene which is “transcriptionally silent,” e.g., not normally expressed, or expressed only at very low levels, may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published in May 16, 1991.

Anti-Tiki1/2 Antibodies

The term “antibody” as used herein refers to an immunoglobulin molecule or immunologically active portion thereof, i.e., an antigen-binding portion. Examples of immunologically active portions of immunoglobulin molecules include F(ab) and F(ab′)2 fragments, which retain the ability to bind antigen. Such fragments can be obtained commercially, or using methods known in the art. For example F(ab)2 fragments can be generated by treating the antibody with an enzyme such as pepsin, a non-specific endopeptidase that normally produces one F(ab)2 fragment and numerous small peptides of the Fc portion. The resulting F(ab)2 fragment is composed of two disulfide-connected Fab units. The Fc fragment is extensively degraded and can be separated from the F(ab)2 by dialysis, gel filtration or ion exchange chromatography. F(ab) fragments can be generated using papain, a non-specific thiol-endopeptidase that digests IgG molecules, in the presence of a reducing agent, into three fragments of similar size: two Fab fragments and one Fc fragment. When Fc fragments are of interest, papain is the enzyme of choice because it yields a 50,00 Dalton Fc fragment; to isolate the F(ab) fragments, the Fc fragments can be removed, e.g., by affinity purification using protein A/G. A number of kits are available commercially for generating F(ab) fragments, including the ImmunoPure IgG1 Fab and F(ab′)2 Preparation Kit (Pierce Biotechnology, Rockford, Ill.). In addition, commercially available services for generating antigen-binding fragments can be used, e.g., Bio Express, West Lebanon, N.H.

The antibody can be a polyclonal, monoclonal, recombinant, e.g., a chimeric, de-immunized or humanized, fully human, non-human, e.g., murine, or single chain antibody. In some embodiments the antibody has effector function and can fix complement. In some embodiments, the antibody has reduced or no ability to bind an Fc receptor. For example, the antibody can be an isotype or subtype, fragment or other mutant, which does not support binding to an Fc receptor, e.g., it has a mutagenized or deleted Fc receptor binding region. The antibody can be coupled to a toxin or imaging agent.

Methods for making suitable antibodies are known in the art. A full-length cancer-related antigen or antigenic peptide fragment thereof can be used as an immunogen, or can be used to identify antibodies made with other immunogens, e.g., cells, membrane preparations, and the like, e.g., E rosette positive purified normal human peripheral T cells, as described in U.S. Pat. Nos. 4,361,549 and 4,654,210.

Methods for making monoclonal antibodies are known in the art. Basically, the process involves obtaining antibody-secreting immune cells (lymphocytes) from the spleen of a mammal (e.g., mouse) that has been previously immunized with the antigen of interest (e.g., a cancer-related antigen) either in vivo or in vitro. The antibody-secreting lymphocytes are then fused with myeloma cells or transformed cells that are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature 256:495 (1975), which is hereby incorporated by reference.

Mammalian lymphocytes are immunized by in vivo immunization of the animal (e.g., a mouse) with an antigen. Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by known techniques, for example, using polyethylene glycol (“PEG”) or other fusing agents (See Milstein and Kohler, Eur. J. Immunol. 6:511 (1976), which is hereby incorporated by reference). This immortal cell line, which is preferably murine, but can also be derived from cells of other mammalian species, including but not limited to rats and humans, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies are also known. Typically, such antibodies can be raised by administering the protein or polypeptide of the present invention subcutaneously to New Zealand white rabbits that have first been bled to obtain pre-immune serum. The antigens can be injected at a total volume of 100 □l per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthanized, e.g., with pentobarbital 150 mg/Kg IV. This and other procedures for raising polyclonal antibodies are disclosed in Harlow, et. al., editors, “Antibodies: A Laboratory Manual” (1988).

In addition to utilizing whole antibodies, the invention encompasses the use of binding portions of such antibodies. Such binding portions include Fab fragments, F(ab′)2 fragments, and Fv fragments. These antibody fragments can be made by conventional procedures, such as proteolytic fragmentation procedures, as described in J. Goding, Monoclonal Antibodies: Principles and Practice, pp. 98-118 (N.Y. Academic Press 1983).

Chimeric, humanized, de-immunized, or completely human antibodies are desirable for applications which include repeated administration, e.g., therapeutic treatment of human subjects.

Chimeric antibodies generally contain portions of two different antibodies, typically of two different species. Generally, such antibodies contain human constant regions and variable regions from another species, e.g., murine variable regions. For example, mouse/human chimeric antibodies have been reported which exhibit binding characteristics of the parental mouse antibody, and effector functions associated with the human constant region. See, e.g., Cabilly et al., U.S. Pat. No. 4,816,567; Shoemaker et al., U.S. Pat. No. 4,978,745; Beavers et al., U.S. Pat. No. 4,975,369; and Boss et al., U.S. Pat. No. 4,816,397, all of which are incorporated by reference herein. Generally, these chimeric antibodies are constructed by preparing a genomic gene library from DNA extracted from pre-existing murine hybridomas (Nishimura et al., Cancer Research, 47:999 (1987)). The library is then screened for variable region genes from both heavy and light chains exhibiting the correct antibody fragment rearrangement patterns. Alternatively, cDNA libraries are prepared from RNA extracted from the hybridomas and screened, or the variable regions are obtained by polymerase chain reaction. The cloned variable region genes are then ligated into an expression vector containing cloned cassettes of the appropriate heavy or light chain human constant region gene. The chimeric genes can then be expressed in a cell line of choice, e.g., a murine myeloma line. Such chimeric antibodies have been used in human therapy.

Humanized antibodies are known in the art. Typically, “humanization” results in an antibody that is less immunogenic, with complete retention of the antigen-binding properties of the original molecule. In order to retain all the antigen-binding properties of the original antibody, the structure of its combining-site has to be faithfully reproduced in the “humanized” version. This can potentially be achieved by transplanting the combining site of the nonhuman antibody onto a human framework, either (a) by grafting the entire nonhuman variable domains onto human constant regions to generate a chimeric antibody (Morrison et al., Proc. Natl. Acad. Sci., USA 81:6801 (1984); Morrison and 01, Adv. Immunol. 44:65 (1988) (which preserves the ligand-binding properties, but which also retains the immunogenicity of the nonhuman variable domains); (b) by grafting only the nonhuman CDRs onto human framework and constant regions with or without retention of critical framework residues (Jones et al. Nature, 321:522 (1986); Verhoeyen et al., Science 239:1539 (1988)); or (c) by transplanting the entire nonhuman variable domains (to preserve ligand-binding properties) but also “cloaking” them with a human-like surface through judicious replacement of exposed residues (to reduce antigenicity) (Padlan, Molec. Immunol. 28:489 (1991)).

Humanization by CDR grafting typically involves transplanting only the CDRs onto human fragment onto human framework and constant regions. Theoretically, this should substantially eliminate immunogenicity (except if allotypic or idiotypic differences exist). However, it has been reported that some framework residues of the original antibody also need to be preserved (Riechmann et al., Nature 332:323 (1988); Queen et al., Proc. Natl. Acad. Sci. USA 86:10,029 (1989)). The framework residues which need to be preserved can be identified by computer modeling. Alternatively, critical framework residues may potentially be identified by comparing known antibody combining site structures (Padlan, Molec. Immun. 31(3):169-217 (1994)). The invention also includes partially humanized antibodies, in which the 6 CDRs of the heavy and light chains and a limited number of structural amino acids of the murine monoclonal antibody are grafted by recombinant technology to the CDR-depleted human IgG scaffold (Jones et al., Nature 321:522-525 (1986)).

Deimmunized antibodies are made by replacing immunogenic epitopes in the murine variable domains with benign amino acid sequences, resulting in a deimmunized variable domain. The deimmunized variable domains are linked genetically to human IgG constant domains to yield a deimmunized antibody (Biovation, Aberdeen, Scotland).

The antibody can also be a single chain antibody. A single-chain antibody (scFV) can be engineered (see, for example, Colcher et al., Ann. N.Y. Acad. Sci. 880:263-80 (1999); and Reiter, Clin. Cancer Res. 2:245-52 (1996)). The single chain antibody can be dimerized or multimerized to generate multivalent antibodies having specificities for different epitopes of the same target protein. In some embodiments, the antibody is monovalent, e.g., as described in Abbs et al., Ther. Immunol. 1(6):325-31 (1994), incorporated herein by reference.

Methods of Treatment

Also described herein are methods of treating a disorder associated with aberrant Wnt signalling, e.g., increased or decreased Wnt signalling.

Disorders Associated with Increased Wnt Signalling

In some embodiments the disorder is associated with increased Wnt signalling, e.g., a disorder associated with unwanted cellular proliferation in which decreased Wnt signaling is associated with better prognosis, e.g., as described herein, by administering a therapeutically effective amount of a secreted form of a Tiki1/2 protein, e.g., as shown in FIG. 12, or a variant thereof, as described herein, that has Wnt inhibitory activity. The treatment should result in an improvement in a parameter of the disorder, e.g., a parameter related to clinical outcome. In some embodiments, the subject is a human, e.g., a human with a disorder associated with unwanted cellular proliferation, e.g., cancer, and the parameter is cellular proliferation, tumor size, metastasis, or survival.

Disorders Associated with Decreased Wnt Signalling

In some embodiments the disorder is associated with reduced Wnt signalling, e.g., a disorder associated with a loss of bone density, e.g., osteoporosis, or a cellular proliferative disorder in which enhanced Wnt signaling is associated with better prognosis, as described herein, by administering a therapeutically effective amount of an inhibitor of a Tiki1/2 protein, e.g., a small molecule identified by a method described herein. The treatment should result in an improvement in a parameter of the disorder, e.g., a parameter related to clinical outcome. In some embodiments, the parameter is bone density, and an improvement would be an increase in or maintenance of bone density over time. In some embodiments, the subject is a human, e.g., a human with loss of bone density, e.g., osteoporosis, and the parameter is maintenance or increase in bone density over time, e.g., with aging. In some embodiments, the subject is a human with a disorder associated with unwanted cellular proliferation, e.g., a cancer in which enhanced Wnt signaling is associated with better prognosis, and the parameter is cellular proliferation, tumor size, metastasis, or survival.

The compounds and methods described herein are useful in the treatment of disorders associated with abnormal apoptotic or differentiative processes, e.g., cellular proliferative disorders or cellular differentiative disorders, e.g., cancer.

Examples of cellular proliferative and/or differentiative disorders include cancer, e.g., carcinoma, sarcoma, metastatic disorders or hematopoietic neoplastic disorders, e.g., leukemias and lymphomas. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit. Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

Other examples of proliferative and/or differentiative disorders include skin disorders. The skin disorder may involve the aberrant activity of a cell or a group of cells or layers in the dermal, epidermal, or hypodermal layer, or an abnormality in the dermal-epidermal junction. For example, the skin disorder may involve aberrant activity of keratinocytes (e.g., hyperproliferative basal and immediately suprabasal keratinocytes), melanocytes, Langerhans cells, Merkel cells, immune cell, and other cells found in one or more of the epidermal layers, e.g., the stratum basale (stratum germinativum), stratum spinosum, stratum granulosum, stratum lucidum or stratum corneum. In other embodiments, the disorder may involve aberrant activity of a dermal cell, e.g., a dermal endothelial, fibroblast, immune cell (e.g., mast cell or macrophage) found in a dermal layer, e.g., the papillary layer or the reticular layer.

Examples of skin disorders include psoriasis, psoriatic arthritis, dermatitis (eczema), e.g., exfoliative dermatitis or atopic dermatitis, pityriasis rubra pilaris, pityriasis rosacea, parapsoriasis, pityriasis lichenoiders, lichen planus, lichen nitidus, ichthyosiform dermatosis, keratodermas, dermatosis, alopecia areata, pyoderma gangrenosum, vitiligo, pemphigoid (e.g., ocular cicatricial pemphigoid or bullous pemphigoid), urticaria, prokeratosis, rheumatoid arthritis that involves hyperproliferation and inflammation of epithelial-related cells lining the joint capsule; dermatitises such as seborrheic dermatitis and solar dermatitis; keratoses such as seborrheic keratosis, senile keratosis, actinic keratosis. photo-induced keratosis, and keratosis follicularis; acne vulgaris; keloids and prophylaxis against keloid formation; nevi; warts including verruca, condyloma or condyloma acuminatum, and human papilloma viral (HPV) infections such as venereal warts; leukoplakia; lichen planus; and keratitis. The skin disorder can be dermatitis, e.g., atopic dermatitis or allergic dermatitis, or psoriasis.

In some embodiments, the disorder is psoriasis. The term “psoriasis” is intended to have its medical meaning, namely, a disease which afflicts primarily the skin and produces raised, thickened, scaling, nonscarring lesions. The lesions are usually sharply demarcated erythematous papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling. Hyperproliferation of keratinocytes is a key feature of psoriatic epidermal hyperplasia along with epidermal inflammation and reduced differentiation of keratinocytes. Multiple mechanisms have been invoked to explain the keratinocyte hyperproliferation that characterizes psoriasis. Disordered cellular immunity has also been implicated in the pathogenesis of psoriasis. Examples of psoriatic disorders include chronic stationary psoriasis, psoriasis vulgaris, eruptive (gluttate) psoriasis, psoriatic erythroderma, generalized pustular psoriasis (Von Zumbusch), annular pustular psoriasis, and localized pustular psoriasis.

One of skill in the art will readily be able to identify those disorders that would benefit from increased or decreased Wnt signaling. For example, it is known that activation of the Wnt/beta-catenin pathway is crucial to the establishment of leukemic stem cells in chronic myeloid leukemia (Ysebaert et al., Leukemia. 2006; 20(7):1211-6). Wnt activation is also important in colorectal, desmoid, ovarian, endometrial, gastric, hepatocellular, kidney (Wilm's tumor), prostate, thyroid, uterine, and lung (e.g., non-small cell lung) cancers. See, e.g., Polakis, Genes Dev. 14:1837-1851 (2000). However, enhanced Wnt signaling is associated with better prognosis of some cancers (such as melanoma (see, e.g., Bachmann et al., Clin Cancer Res 11:8606-8614 (2005); Chien et al., PNAS 106:1193-1198 (2009); Kageshita et al., Br J Dermatol 145:210-216 (2001); and Maelandsmo et al., Clin Cancer Res 9:3383-3388 (2003)) and medulloblastoma (see, e.g., Ellison et al., J Clin Oncol 23:7951-7957 (2005); Fattet et al., J Pathol 218:86-94 (2009); and Kool et al., Plos One 3, e3088 (2008)), possibly due to enhanced differentiation by Wnt in these cancers (see also Dejmek et al., Cancer Res 65:9142-9146 (2005); and Lame and Delmas, Frontiers in Bioscience 11:733-742 (2006)). Other diseases associated with aberrant Wnt signaling may also benefit from treatment with the compounds and methods described herein, see, e.g., Moon et al., Nature Reviews Genetics 5, 691-701 (2004).

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Methods of Screening

Small chemical compound inhibitors are attractive drug candidates for treating osteoporosis or disorders associated with unwanted cellular proliferation, e.g., cancer. However, the known Wnt antagonists such as Sclerostin, DKK1, and SFRP1 function through protein-protein interactions and their inhibition via small molecules is difficult to achieve. In contrast, Tiki1/2 (e.g., Tiki2) appear to be ideal therapeutic targets for several reasons. First, Tiki2−/−, or even Tiki2+/−, mice show increased bone mass, suggesting that inhibition, or partial inhibition, of Tiki2 may benefit osteoporosis treatment. Second, Tiki2−/− mice are viable, suggesting that long-term Tiki inhibition may have minimal adverse effects. Third, Tiki proteins act as enzymes, which in general are ‘drugable’, i.e., suitable for inhibition by chemical compounds. Therefore the present invention includes methods of identifying small molecule inhibitors for Tiki1/2 for treatment of osteoporosis, or small molecule activators or mimics of Tiki1/2 for treatment of disorders associated with unwanted cellular proliferation, e.g., cancer.

Included herein are methods for screening test compounds, e.g., polypeptides, polynucleotides, inorganic or organic large or small molecule test compounds, to identify agents useful in the treatment of disorders associated with loss of bone density, e.g., osteoporosis, or disorders associated with unwanted cellular proliferation, e.g., cancer.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The test compounds can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of small molecule libraries are commercially available. A number of suitable small molecule test compounds are listed in U.S. Pat. No. 6,503,713, incorporated herein by reference in its entirety.

Libraries screened using the methods of the present invention can comprise a variety of types of test compounds. A given library can comprise a set of structurally related or unrelated test compounds. In some embodiments, the test compounds are peptide or peptidomimetic molecules. In some embodiments, the test compounds are nucleic acids.

In some embodiments, the test compounds and libraries thereof can be obtained by systematically altering the structure of a first test compound, e.g., a first test compound that is structurally similar to a known natural binding partner of the target polypeptide, or a first small molecule identified as capable of binding the target polypeptide, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in one embodiment, a general library of small molecules is screened, e.g., using the methods described herein.

In some embodiments, a test compound is applied to a test sample, e.g., a cell or living tissue or organ, e.g., a bone cell or an osteoclast, or a tumor cell (e.g., a primary or cultured tumor cell), and one or more effects of the test compound is evaluated. In a cultured or primary cell for example, the ability of the test compound to modulate Wnt signalling is evaluated.

In some embodiments, the test sample is, or is derived from (e.g., a sample taken from) an in vivo model of a disorder as described herein. For example, an animal model, e.g., a rodent such as a rat, can be used, and an effect on bone density can be evaluated. Alternatively or in addition, an animal model of a disorder associated with unwanted cellular proliferation can be used, e.g., a normal, xenograft or genetic mutant rodent model that has or is prone to developing tumors or other conditions associated with unwanted cellular proliferation as described herein.

Methods for evaluating each of these effects are known in the art. For example, ability to modulate expression of a protein (e.g., Tiki or Wnt) can be evaluated at the gene or protein level, e.g., using quantitative PCR or immunoassay methods. In some embodiments, high throughput methods, e.g., protein or gene chips as are known in the art (see, e.g., Ch. 12, Genomics, in Griffiths et al., Eds. Modern genetic Analysis, 1999, W.H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect an effect on Tiki expression. Ability to modulate Tiki signaling via the Wnt pathway can be evaluated, e.g., using methods known in the art. Bone density can also be evaluated using methods known in the art, e.g., as described herein. Cellular proliferation rates can also be evaluated using methods known in the art.

Methods for evaluating Tiki activity, e.g., Tiki inhibition of Wnt, are described herein. For example, a test compound can be evaluated in cells expressing both Tiki1/2 and Wnt, e.g., Wnt3A, Wnt activity is evaluated, e.g., by Wnt-responsive luciferase reporter assays. Such assays use a reporter gene such as the fruitfly luciferase, whose expression is under the control of Wnt-responsive DNA elements (WREs) (Molenaar et al., Cell 86 (1996), pp. 391-399. Korinek et al., Science 1997. 275, 1784-1787. Major et al., Science. 2007. 316:1043-6). The specificity of the effect can be confirmed by comparison of the activity of the test compound in cells that do not express Tiki1/2; a test compound that affects Wnt activity in the presence of Tiki1/2, but does not affect Wnt activity in the absence of Tiki1/2, can be considered to be a specific modulator of Tiki1/2. In some embodiments, an assay of Wnt modification, e.g., an assay for Tiki1/2-associated enzyme activity that deacylates/depalmitoylates WNT3A, can be used (e.g., using assays known in the art and/or described herein, see the Examples, below). In addition, binding experiments to detect specific binding of a test compound to Tiki1/2 can also be used to detect specificity (Huang et al., Nature Chemical Biology 2009).

A test compound that has been screened by a method described herein and determined to modulate Tiki activity, e.g., in an in vitro screen, can be considered a candidate compound. A candidate compound that has been screened, e.g., in an in vivo model of a disorder, e.g., loss of bone density, e.g., osteoporosis, or cellular proliferative disorders, and determined to have a desirable effect on the disorder, e.g., on one or more symptoms of the disorder, can be considered a candidate therapeutic agent. Candidate therapeutic agents, once screened in a clinical setting, are therapeutic agents. Candidate compounds, candidate therapeutic agents, and therapeutic agents can be optionally optimized and/or derivatized, and formulated with physiologically acceptable excipients to form pharmaceutical compositions.

Thus, test compounds identified as “hits” (e.g., test compounds that increase or maintain bone density or affect Tiki/Wnt signaling) in a first screen can be selected and systematically altered, e.g., using rational design, to optimize binding affinity, avidity, specificity, or other parameter. Such optimization can also be screened for using the methods described herein. Thus, in one embodiment, the invention includes screening a first library of compounds using a method known in the art and/or described herein, identifying one or more hits in that library, subjecting those hits to systematic structural alteration to create a second library of compounds structurally related to the hit, and screening the second library using the methods described herein.

Test compounds identified as hits can be considered candidate therapeutic compounds, useful in treating disorders associated with loss of bone density, e.g., osteoporosis, or cellular proliferative disorders. A variety of techniques useful for determining the structures of “hits” can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disorder described herein.

Test compounds identified as candidate therapeutic compounds can be further screened by administration to an animal model of a disorder associated with loss of bone density, e.g., osteoporosis, or a cellular proliferative disorder, as described herein. The animal can be monitored for a change in the disorder, e.g., for an improvement in a parameter of the disorder, e.g., a parameter related to clinical outcome. In some embodiments, the parameter is bone density, and an improvement would be an increase in or maintenance of bone density over time. In some embodiments, the subject is a human, e.g., a human with loss of bone density, e.g., osteoporosis, and the parameter is maintenance or increase in bone density over time, e.g., with aging. In some embodiments, the subject is a human, e.g., a human with a disorder associated with unwanted cellular proliferation, e.g., cancer, and the parameter is cellular proliferation, tumor size, metastasis, or survival.

Compounds identified by methods described herein are also useful in the treatment of disorders associated with abnormal apoptotic or differentiative processes, e.g., cellular proliferative disorders or cellular differentiative disorders, e.g., cancer.

Pharmaceutical Compositions and Methods of Administration

The methods described herein include the manufacture and use of pharmaceutical compositions, which include as active ingredients compounds identified by a method described herein as active ingredients. Also included are the pharmaceutical compositions themselves. In some embodiments, the active ingredient is a truncated or soluble form of Tiki1/2, e.g., as shown in FIG. 12, or a variant thereof.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### EXAMPLES

The invention is further described in the following examples, which do not limit the scope of the invention described in the claims.

### Example 1

### Expression Cloning of Tiki1

A functional cDNA expression screen was performed using arrayed Xenopus cDNAs to identify novel genes involved in AP patterning and Wnt signaling. Procedures for embryo manipulation, reverse transcription PCR and in situ hybridization were performed as previously described (Tamai, K. et al., Nature 407, 530-535 (2000)). The Arrayed Xenopus tropicalis full length cDNAs were used for the screening as performed previously (Kato, Y. et al., Nature 418, 641-646 (2002)). cDNA pools (in the CS107 vector) that contain 96 independent clones were transcribed in vitro using SP6 polymerase, and the resulting mRNA pools were injected into the dorsal side of the embryo at 4-cell stage at the dose of 10 ng RNA/embryo. Injected embryos were scored at stage 30-31 for enlarged or diminished anterior development. The positive pools were divided into smaller pools and the same process was repeated until a single cDNA was identified.

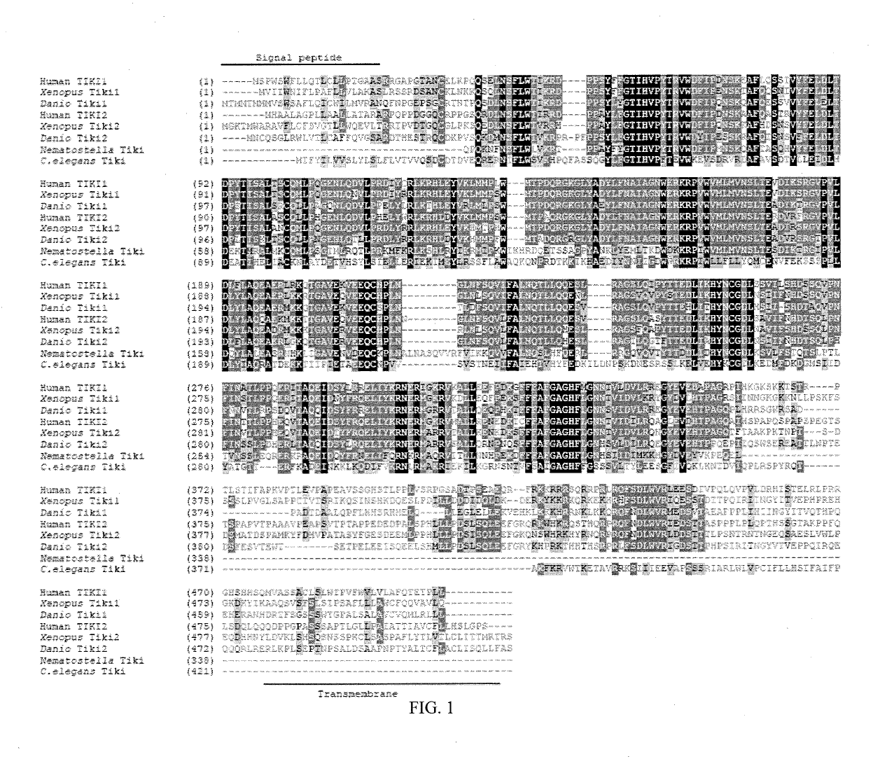

One of the identified cDNAs caused dramatic head enlargement in embryos upon overexpression and was named Tiki1 (“Tiki” refers to a large headed humanoid carving in Polynesian mythology). The Tiki1 gene encodes a putative type I transmembrane protein of 508 residues, harboring a leading amino-terminal signal peptide, a large ectodomain, and a transmembrane domain at the carboxyl terminus (FIG. 1).

This original Xenopus Tiki1 cDNA in pCS107 contained the full coding region and parts of 5′ and 3′ UTR. Human Tiki1 and Tiki2 genes were amplified from a human cell line via RT-PCR and cloned into pCS2+. Also created in pCS2+ were: HA-Tiki1 (which has a HA tag at the amino terminus after a heterologous signal peptide from human Fz5). HA-Tiki1N (which has the carboxyl terminal transmembrane domain deleted), HA-Tiki1/2, HA-Tiki1/2N, Tiki1-FLAG (which contains a part of endogenous 5′ UTR and a FLAG tag at the carboxyl terminus), Tiki2N-FLAG-6×HIS (which is the extracellular domain of Tiki2 fused with a carboxyl FLAG tag and a 6×HIS tag and was used for tandem affinity purification (TAP)), Krm2N-FLAG-6×HIS, and Tiki2N□-FLAG-6×HIS (which is the extracellular domain of Tiki2 with a deletion from residue 35 to 222). The HA-Tiki1 and 2 were subcloned into pBABE-puro vector (Addgene plasmid 1764) for retroviral expression.

Tiki1 is the founding member of a new protein family conserved from invertebrates to vertebrates, and is also found in the genome of sea anemone Nematostella thus is likely in the common ancestor before invertebrates and vertebrates diverged. Most vertebrates including Xenopus, zebrafish and human have two genes, Tiki1 and Tiki2 (FIG. 1). Tiki proteins do not harbor any functional motifs known to metazoans, but intriguingly exhibit a conserved ectodomain of about 340 residues, referred to here as the “Tiki domain,” which shares significant homology (approximately 25% identity/40% similarity) with several putative bacterial proteins of the gumN superfamily, all of unknown function.

Tiki1 was expressed in human HEK293T and HeLa cells as follows. HEK293T, and HeLa cells (from ATCC) were maintained in Dulbecco's modified Eagle's medium (DMEM; Invitrogen) supplemented with 10% fetal bovine serum (FBS) and 1% penicillin-streptomycin-glutamine (PSA; Invitrogen). Lipofectamine 2000 (Invitrogen) or Fugene6 (Roche) was used for all cell transfections. Tiki1 was detected at the plasma and internal secretory membranes but not in conditioned media (CM), indicating that the protein is membrane-bound and not secreted in those cells.

### Example 2

### Tiki1 Antagonizes Wnt Function

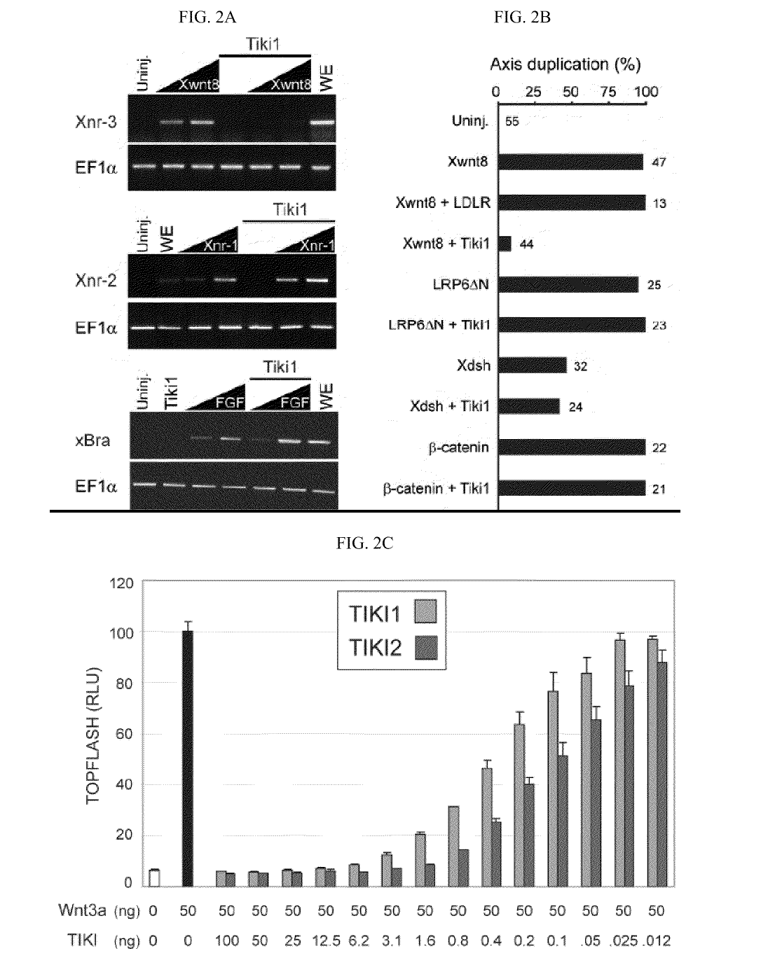

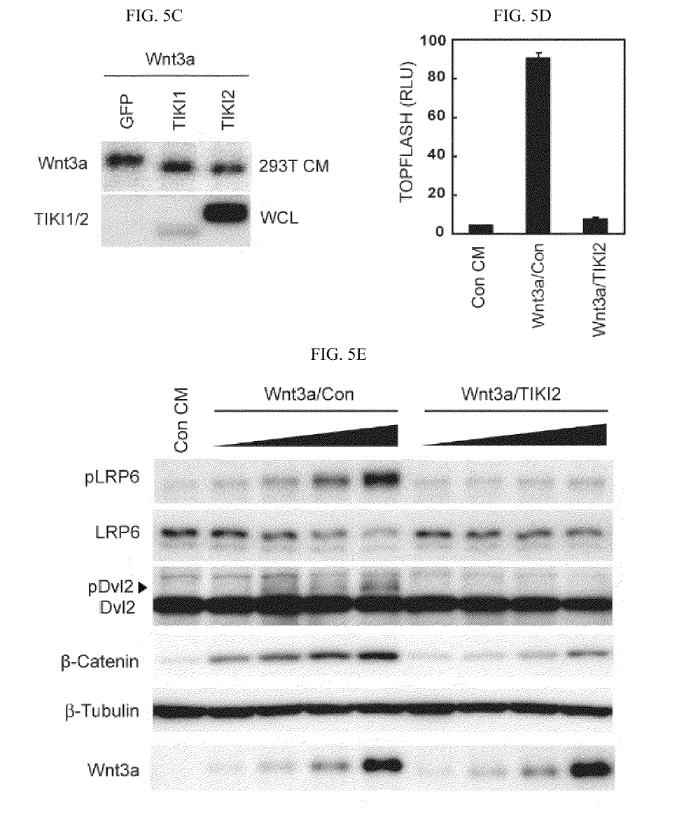

The enlarged head phenotype by Tiki1 overexpression is indistinguishable from that by overexpression of Dkk1, a Wnt/LRP6 antagonist (MacDonald, B. T. et al., Dev Cell 17, 9-26 (2009); Glinka, A. et al., Nature 391, 357-362 (1998)). Indeed Tiki1 antagonized Wnt function in embryos, and inhibited axis duplication and induction of Xnr3, a Wnt/beta-catenin target gene, by Xwnt8, but not by beta-catenin or another Wnt downstream component Dishevelled, or by a constitutively activated LRP6ΔN (Tamai, K. et al., Mol Cell 13, 149-156 (2004)) (FIG. 2A, top panel, and 2B). Thus Tiki1 likely acts at or upstream of Wnt receptors. Tiki1 did not affect signaling by other secreted factors such as Nodal or bFGF (basic fibroblast growth factor) in embryos (FIG. 2A, middle and bottom panels) attesting its specificity as a Wnt antagonist. Xenopus Tiki2, and human Tiki1 and Tiki2 also behaved as Wnt antagonists in Xenopus embryos, HEK293T cells (FIG. 2C) and mouse L cells (FIG. 5A).

### Example 3

### Organizer-Specific Expression of Tiki1

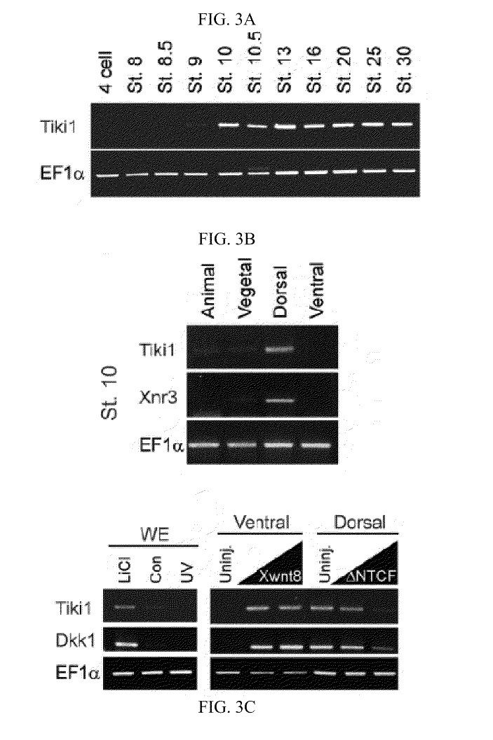

Tiki1 expression was investigated during Xenopus early development. RT-PCR revealed that Tiki1 mRNA becomes detectable at stage 9 and is prominently expressed from stage 10 (early gastrula) to stage 30 (tadpole) (FIG. 3A), and interestingly is restricted to the dorsal segment of the gastrula embryo (FIG. 3B). Whole mount in situ hybridization showed that at stage 10 Tiki1 is expressed exclusively in the forming dorsal blastopore lip, the Spemann-Mangold Organiser (SO), in a pattern that is similar to that of Dkk1. Longitudinal hemi-sectioning of the stage 10.5 embryo revealed that Tiki1 is excluded from the dorsal margin of the SO and overlapped with Dkk1 expression in the prechordal mesoderm and endomesoderm. At neurula stages Tiki1 and Dkk1 are strongly expressed in the anterior domain of the prospective prechordal plate. By the closure of the blastopore Tiki1 expression domain becomes distinct from that of Dkk1 as Tiki1 is more restricted to the midline anteriorly, while Dkk1 displays a wing-shaped expression domain straddling Tiki1. At the late neurula stage Tiki1 is expressed in the midline anterior to the tip of the notochord in cells of the endoderm and overlying neural ectoderm while Dkk1 has a more broad expression pattern in the prospective ventral forebrain. Therefore Tiki1 is zygotically and specifically expressed in the SO, in particular in the head organizer region responsible for anterior patterning.

SO formation requires maternal Wnt/β-catenin signaling (De Robertis, E. M. et al., Annu Rev Cell Dev Biol 20, 285-308 (2004); Harland, R. et al., Annu Rev Cell Dev Biol 13, 611-667 (1997)), which we found is necessary and sufficient for Tiki1 (and Dkk1) expression. Indeed ectopic Xwnt8 induced Tiki1 and Dkk1 expression ventrally, whereas a dominant negative TCF (T cell factor, a DNA-binding transcription factor that mediates signaling by beta-catenin, ref. (Molenaar, M. et al., Cell 86, 391-399 (1996))), ΔNTCF, suppressed the endogenous Tiki1 and Dkk1 dorsal expression (FIG. 3C). Furthermore LiCl, which stabilizes β-catenin thus dorsalizes embryos (Harland, R. et al., Annu Rev Cell Dev Biol 13, 611-667 (1997)), induced Tiki1 and Dkk1 expression, while ultraviolet irradiation, which through microtubule disruption causes defective maternal Wnt signaling thus ventralizes embryos (Harland, R. et al., Annu Rev Cell Dev Biol 13, 611-667 (1997)), suppressed Tiki1 and Dkk1 expression (FIG. 3C). Thus Tiki1 exhibits similar regulation as Dkk1 in the dorsal organizer.

### Example 4

### Requirement of Tiki1 in Head Development

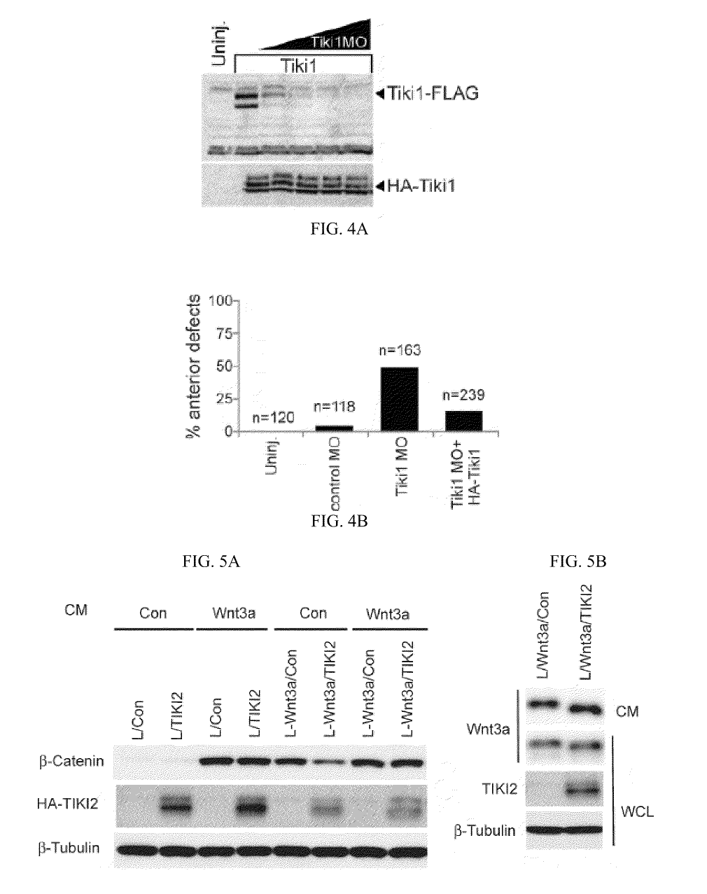

Loss-of-function experiments were performed via Tiki1 protein depletion using a morpholino antisense oligonucleotide against Tiki1 (Tiki1MO), 5′-CCAAATGATTACCATCATAGCTCAG-3′ (SEQ ID NO:13), which specifically blocked the synthesis of the exogenously expressed Tiki1 (Tiki1-FLAG, tagged) in embryos (FIG. 4A). Tiki1MO was unable to block the synthesis of HA-Tiki1 (tagged), whose mRNA was engineered to lack the MO complementary sequence, attesting Tiki1MO targeting specificity (FIG. 4A). 20 ng of Tiki1MO was injected into two dorsal blastomeres of 8-cell stage embryos. Strikingly roughly 50% of Tiki1 MO-injected embryos developed anterior defects exhibiting loss of forebrain structures, diminished or loss of the cement gland and eyes fused at the midline reminiscent of cyclopia (FIG. 4B). Co-injection of HA-Tiki1 mRNA rescued Tiki1MO phenotypes (FIG. 4B). Supporting a role of Tiki1 in Wnt inhibition during head formation, Dkk1 mRNA injection also rescued significantly Tiki1 MO anterior deficiency phenotypes.

In addition, injection of Tiki1MO in two dorsal blastomeres of 8-cell stage embryos suppressed the expression of head organizer genes including Goosecoid, Lim1, Otx2, and Dkk1 in gastrula stage embryos (stage 10.5) (Table 1). Tiki1 MO also inhibited the expression of the neuralizing/dorsalizing organizer gene Chordin, but not of other SO or dorsally expressed genes Xnr3 and Xnot (Table 1), which are not involved in head organizer function. Tiki1 MO also reduced Goosecoid expression in prechordal plate, the descendent of the head organizer, at the neurula stage (Table 1). HA-Tiki1 mRNA injection rescued the effect of Tiki1MO on all head organizer genes examined (Table 1). Control MO-injected embryos showed normal expression of all markers examined, similar to uninjected embryos (Table 1). These results show that Tiki1 is specifically required for head organizer formation.

TABLE 1
Whole embryos displaying the described change in gene expression:

| | Uninjected | | | | | | | | | | | | | | | |
|---|---|---|---|---|---|---|---|---|---|---|---|---|---|---|---|---|
| | | Percentage (%) | | | Control MO | | | | Tiki1 MO | | | | Tiki1 MO + HA-Tiki1 | | | |
| | | Ab- | | | | Percentage (%) | | | | Percentage (%) | | | | Percentage (%) | | |
| Gene | n | sent | Reduced | NC* | n | Absent | Reduced | NC* | n | Absent | Reduced | NC* | n | Absent | Reduced | NC* |
| Chd | 25 | 0 | 0 | 100 | 34 | 0 | 3 | 97 | 39 | 0 | 36 | 64 | 31 | 0 | 23 | 77 |
| Gsc | 29 | 0 | 10 | 90 | 30 | 0 | 3 | 97 | 45 | 0 | 66 | 34 | 31 | 0 | 23 | 77 |
| Lim1 | 17 | 0 | 0 | 100 | 40 | 0 | 0 | 100 | 35 | 28 | 54 | 18 | 37 | 3 | 19 | 78 |
| Otx2 | 35 | 0 | 0 | 100 | 37 | 5 | 3 | 92 | 40 | 7 | 60 | 33 | 33 | 0 | 30 | 70 |
| Dkk1 | 31 | 0 | 0 | 100 | 40 | 0 | 3 | 97 | 47 | 25 | 40 | 35 | 31 | 6 | 16 | 78 |
| Xnr3 | 24 | 0 | 4 | 96 | 31 | 0 | 0 | 100 | 27 | 0 | 7 | 93 | 42 | 0 | 5 | 95 |
| Xnot | 20 | 0 | 0 | 100 | 30 | 0 | 0 | 100 | 32 | 0 | 13 | 87 | 39 | 0 | 0 | 100 |
| Gsc | 38 | 0 | 0 | 100 | 31 | 0 | 0 | 100 | 45 | 35 | 32 | 33 | 38 | 13 | 0 | 87 |
| st.13 | | | | | | | | | | | | | | | | |

*NC = No Change

The specific expression in and the requirement for the formation of the head organizer imply Tiki1 function in anterior neural patterning. Tiki1MO was injected with fluorescein dextran (FLD, as a lineage tracer) into one dorsal-animal blastomeres at the 8-cell stage and analyzed expression of anterior neural markers at stage 16. Tiki1MO suppressed the expression of, in the injected half of the embryo, forebrain marker BF1 and Otx2 (Table 2). Tiki1MO also reduced the expression of the midbrain marker En-2, but did not affect the posterior neural marker Hoxb9 (Table 2). HA-Tiki1 mRNA injection rescued the expression of BF1, Otx2, and En2 in Tiki1MO-injected embryos (Table 2). These results show that Tiki1 function is required for anterior neural patterning, consistent with its essential role in the head organizer.

TABLE 2
Whole embryos displaying the described change in gene expression

| | | Percentage (%) | | | | | Percentage (%) | | | |
|---|---|---|---|---|---|---|---|---|---|---|
| Gene | n | Absent | Reduced | NC* | increased | n | Absent | Reduced | NC* | increased |
| | Control MO | | | | | Tiki1 MO | | | | |
| BF1 | 45 | 0 | 5 | 95 | 0 | 35 | 63 | 23 | 14 | 0 |
| Otx2 | 37 | 0 | 0 | 100 | 0 | 42 | 35 | 30 | 35 | 0 |
| En-2 | 45 | 0 | 2 | 98 | 0 | 35 | 40 | 25 | 35 | 0 |
| HoxB9 | 23 | 0 | 0 | 100 | 0 | 21 | 0 | 0 | 100 | 0 |
| | Control MO + HA-Tiki1 | | | | | Tiki1 MO + HA-Tiki1 | | | | |
| BF1 | 37 | 0 | 0 | 22 | 78 | 48 | 0 | 23 | 4 | 73 |
| Otx2 | 28 | 0 | 0 | 14 | 86 | 45 | 0 | 13 | 11 | 76 |
| En-2 | 37 | 0 | 0 | 54 | 46 | 48 | 0 | 15 | 45 | 40 |
| HoxB9 | — | — | — | — | — | 25 | 0 | 0 | 100 | 0 |

*NC = No Change

### Example 5

### Wnt Inactivation by Tiki Proteins

As Tiki proteins are related to bacterial proteins of unknown function, it was speculated that Tiki may antagonize Wnt signaling through a novel mechanism, possibly in an enzymatic fashion. Human Tiki2 was stably expressed in L cells or Wnt3a-expressing L cells (human Tiki1 was poorly expressed in L cells). Intriguingly Tiki2 expression inhibited beta-catenin stabilization in Wnt3a-producing cells, but not in L-cells treated with Wnt3a CM (FIG. 5A). In fact Wnt3a CM restored beta-catenin stabilization in Wnt3a-expressing cells that also expressed Tiki2 (FIG. 5A). These results suggest that Tiki2 may inhibit Wnt3a production or activity. Wnt3a produced in L cells in the presence of Tiki2 was secreted into CM in an indistinguishable manner as in the absence of Tiki2, but interestingly exhibited faster electrophoretic migration both prior to (as detected in whole cell lysates) and after secretion (in CM) (FIG. 5B). Similarly in HEK293T cells Wnt3a was secreted into CM normally but exhibited faster electrophoretic mobility in the presence of either Tiki2 or Tiki1 even though ectopic Tiki1 expression was much poorer than that of Tiki2 (FIG. 5C). Importantly Wnt3a secreted from Tiki-expressing cells exhibited minimal activity, as it neither activated the Wnt-responsive reporter expression (FIG. 5D), nor induced LRP6 or Dishevelled phosphorylation or beta-catenin stabilization (FIG. 5E). Importantly Wnt3a secreted from Tiki2-expressing cells failed to bind to either Fz or LRP6 as examined using the extracellular domain of mouse Fz8 (mFz8CRD-IgG) or LRP6 (LRP6N-IgG) (Tamai, K. et al., Nature 407, 530-535 (2000)) (FIG. 5F). These data suggest that Tiki proteins result in a posttranslational modification of Wnt3a that minimizes Wnt3a activity but not secretion.

### Example 6

### Wnt Deacylation/Depalmitoylation by Tiki Proteins

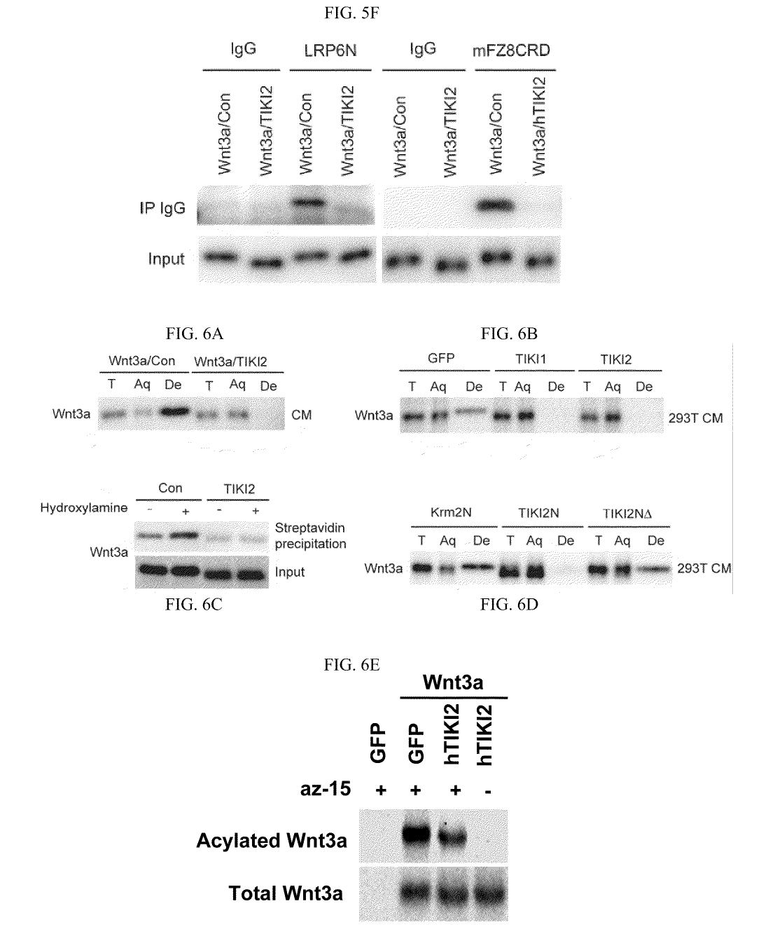

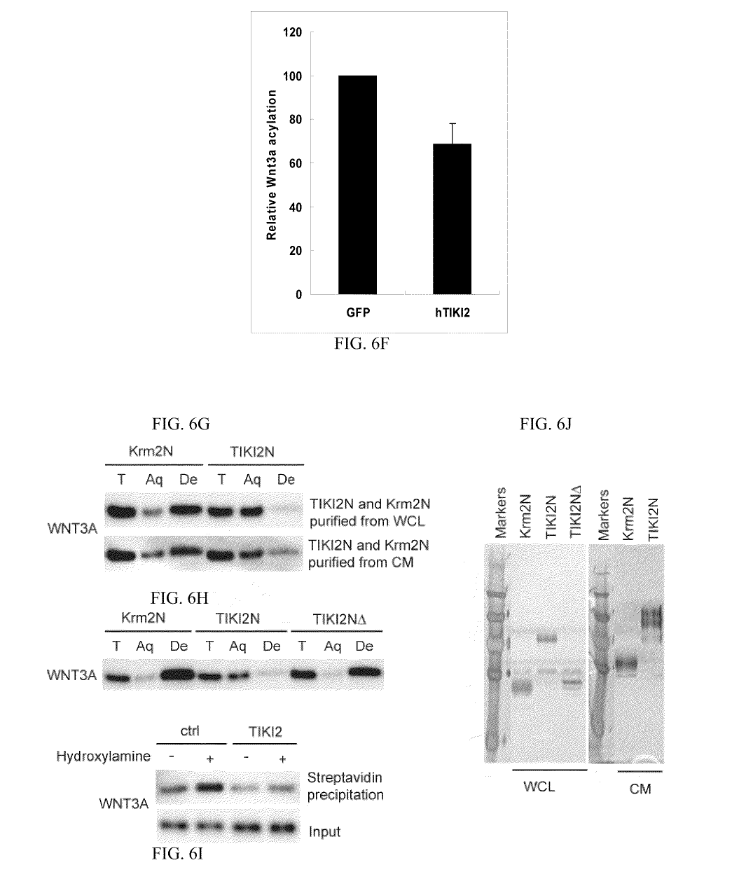

C77 palmitoylation is required for Wnt3a activity (Willert, K. et al., Nature 423, 448-452 (2003)) and binding to both Fz and LRP6 (Komekado, H. et al., Genes Cells 12, 521-534 (2007); Cong, F. et al., Development 131, 5103-5115 (2004)), while S209 acylation is critical for Wnt3a secretion (Takada, R. et al., Dev Cell 11, 791-801 (2006)). Thus Tiki proteins may affect C77 palmitoylation but not S209 acylation. The palmitate adduct at C77 is the primary contributor to Wnt3a hydrophobicity because palmitoylated Wnt3a is partitioned in the Triton X-114 detergent phase whereas Wnt3a lacking the palmitate is soluble in the aqueous phase (Willert, K. et al., Nature 423, 448-452 (2003); Komekado, H. et al., Genes Cells 12, 521-534 (2007)). Contrary to Wnt3a secreted from control cells, which was mainly in the detergent phase, Wnt3a from Tiki2-expressing L cells or from Tiki1- or Tiki2-expressing HEK293T cells partitioned exclusively in the aqueous phase (FIGS. 6A-B), suggesting that Tiki expression may cause a loss of palmitate at C77. Glycosylation, which is the other major form of post-translational modifications of Wnt3a, does not contribute to its hydrophobic behavior since fully deglycosylated Wnt3a partitions in the detergent phase in an identical manner as glycosylated Wnt3a (Komekado, H. et al., Genes Cells 12, 521-534 (2007)).

To further demonstrate Wnt depalmitoylation, an acyl-biotinyl exchange (ABE) assay was performed in which palmitate adduct attached to a cysteine residue via the thioester bond can be replaced by a thiol-specific biotinylation reagent in the presence of hydroxylamine (Drisdel, R. C. et al., Biotechniques 36, 276-285 (2004); Roth, A. F. et al., Cell 125, 1003-1013 (2006)). Biotinylation of Wnt3a secreted in the presence of Tiki2 was much reduced compared to that of Wnt3a from control cells (FIG. 6C). This reduction of available thioester linkage indicates depalmitoylation of Wnt3a at a cysteine residue, e.g., C77, in the presence of Tiki2.

Metabolic labeling was performed to quantify Wnt3a acylation. Wnt3a co-expressed with GFP (control) or hTiki2 in HEK293T cells was metabolically labeled with an azido-palmitic acid analog (az-15) and [³⁵S]-Methionine/Cysteine. Wnt3a secreted in the culture medium was immunoprecipitated and subjected to click chemistry reaction using the Rhodamine-labeled ALK reagent (ALK-Rho), which reacts with az-15 (Charron G et al., JACS131, 4967, 2009). The fluorescent signal indicates the acylation amount of Wnt3a whereas ³⁵S signal indicates the total amount of Wnt3a protein (FIG. 6E). These experiments show that hTiki2 reduces Wnt3a acylation by about 31% (FIG. 6F). Note that Wnt3a has two and perhaps more acylation sites, and our other data suggest that Tiki only removes/deacylates one of these, likely C77 palmitoylation. Thus the maximal reduction of acylation by Tiki2 is expected to be 50% if the deacylation is complete and there are only two acylation sites. The 31% reduction we observed is consistent with the possibility of three acylation sites, although it is also possible that there are only two acylation sites in Wnt3a, and Tiki2 deacylation of one of these two sites was incomplete in our experiment.

### Example 7

### Tiki is Likely a Wnt Deacylase

These data suggest that Tiki is either an inhibitor of the C77 acyltransferase or is a deacylase, i.e., an acylthioesterase that depalmitoylates Wnt3a. Recombinant human Tiki2N, which is the ectodomain harboring the conserved Tiki domain (FIG. 6J), was purified, via tandem affinity procedures from HEK293T cell extracts or CM. Recombinant human WNT3A protein was palmitoylated and mostly partitioned in the detergent phase, but upon in vitro incubation with Tiki2N became segregated in the aqueous phase thus likely depalmitoylated at C77 (FIG. 6G). WNT3A remained in the detergent phase upon in vitro incubation with either a control recombinant protein, KrmN, which corresponds to the ectodomain of the transmembrane Kremen protein (Mao, B. et al., Nature 417, 664-667 (2002)), or Tiki2NΔ, which is a deletion mutant lacking a segment of the Tiki domain and is inactive in modifying WNT3A when coexpressed in HEK293T cells (FIGS. 6F-H). Further, in the ABE assay WNT3A was biotinylated to a much less extent after incubation with purified Tiki2N (FIG. 6I), suggesting that the acylthioester bond at C77 in WNT3A had been hydrolyzed in vitro by Tiki2N These results suggest that Tiki2 has, or at least is associated with, an acylthioesterase activity that depalmitoylates WNT3A.

### Example 8

### Tiki Acts in Both Wnt-Producing and -Responding Cells

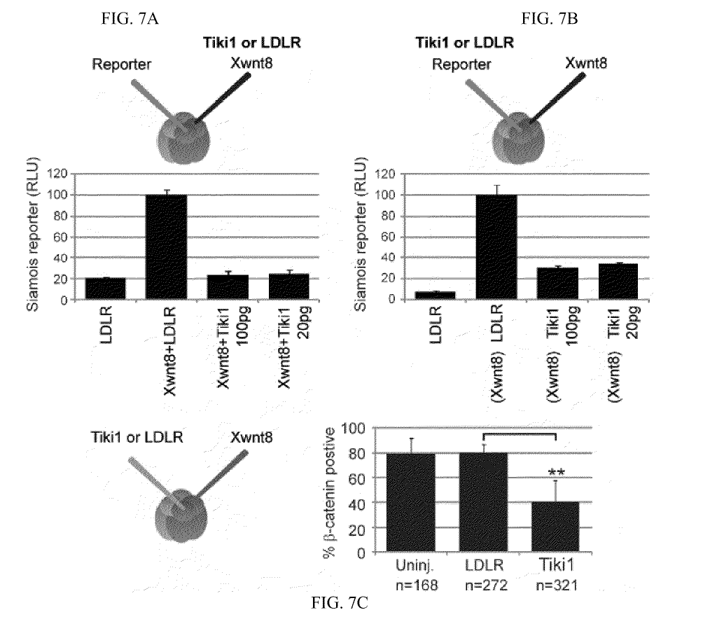

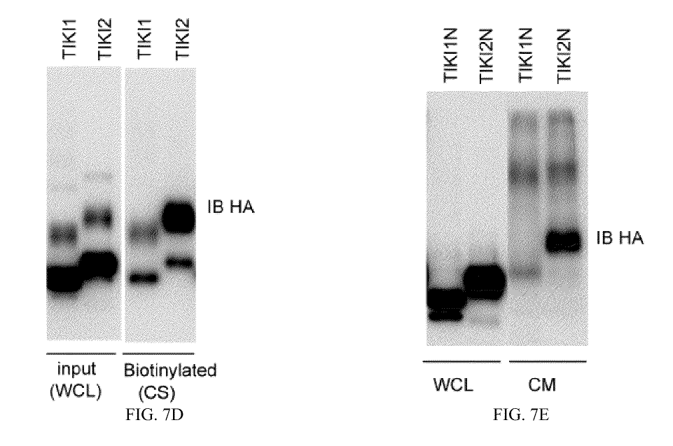

Although Tiki showed strong inhibitory effect in Wnt-producing, but not Wnt-responding L cells (FIG. 5A), it was possible that excessive Wnt3a proteins added in the latter condition might have overwhelmed Tiki function. Therefore it was further investigated whether Tiki1 was able to block Wnt/beta-catenin signaling in Wnt-producing and -responding cells in Xenopus embryos. First a Wnt-responsive reporter assay was performed. At the 8-cell stage two animal blastomeres were injected separately with Xwnt8 mRNA or S01234-luciferase DNA (FIG. 7A), which is driven by the promoter of the Xenopus Siamois gene, a direct Wnt target gene (Brannon, M. et al., Genes Dev 11, 2359-2370 (1997)). Xwnt8 induced luciferase expression from the reporter (FIG. 7A), but not from a control reporter in which all TCF-binding sites were mutated (Brannon, M. et al., Genes Dev 11, 2359-2370 (1997)). Co-injection of Xwnt8 with Tiki1, but not with the control LDLR, inhibited the reporter expression (FIG. 7A). Therefore Tiki1 could inhibit Wnt activity in Wnt-producing cells in embryos as seen in L cells. The co-injection scheme was then altered by injecting Xwnt8 in one animal blastomere and co-injection of Tiki1 or LDLR mRNA together with the 501234-luciferase reporter in a neighboring animal blastomere (FIG. 7B). Under these conditions, Tiki1, but not LDLR, also inhibited Xwnt8-induced reporter expression (FIG. 7B).

Secondly Wnt-induced nuclear beta-catenin accumulation was monitored. Xwnt8 plus RFP (red fluorescent protein) mRNA was injected into a single blastomere of 8-cell stage embryos, and injected Tiki1 or LDLR mRNA together with fluorescein dextran (FLD) into a neighboring blastomere (FIG. 7C), and nuclear β-catenin was examined in animal cap (AC) cells at stage 9. In confocal microscopy Xwnt8-expressing cells were traced by RFP (red), whereas Wnt-responding cells that expressed Tiki1 or LDLR were traced by FLD (green), and naive cells descended from uninjected blastomeres lacked green or red fluorescence. Nuclear beta-catenin was observed in Xwnt8-expressing cells due to autocrine signaling, and in cells that expressed LDLR in response to paracrine Wnt signaling (FIG. 7C). By contrast, much fewer Tiki1-expressing AC cells exhibited nuclear beta-catenin (FIG. 7C). Quantification showed that 80% of LDLR-expressing and naive AC cells, but only 40% of Tiki1-expressing cells, displayed nuclear beta-catenin (FIG. 7C). These results together suggest that Tiki1 also functions in Wnt-responding cells to block signaling, likely via acting at the plasma membrane to inactivate Wnt proteins. These results are consistent with our observation that Tiki1 is likely a Wnt-deacylase via its ectodomain and could be found at the plasma membrane (FIG. 7D-E).
1. Logan, C. Y. & Nusse, R. The Wnt signaling pathway in development and disease. Annu Rev Cell Dev Biol 20, 781-810 (2004).
2. Moon, R. T., Kohn, A. D., De Ferrari, G. V. & Kaykas, A. WNT and beta-catenin signalling: diseases and therapies. Nat Rev Genet. 5, 691-701 (2004).
3. Clevers, H. Wnt/beta-catenin signaling in development and disease. Cell 127, 469-480 (2006).
4. Zecca, M., Basler, K. & Struhl, G. Direct and long-range action of a wingless morphogen gradient. Cell 87, 833-844 (1996).
5. Strigini, M. & Cohen, S. M. Wingless gradient formation in the Drosophila wing. Curr Biol 10, 293-300 (2000).
6. Hausmann, G., Banziger, C. & Basler, K. Helping Wingless take flight: how WNT proteins are secreted. Nat Rev Mol Cell Biol 8, 331-336 (2007).
7. Ching, W., Hang, H. C. & Nusse, R. Lipid-independent secretion of a Drosophila Wnt protein. J Biol Chem 283, 17092-17098 (2008).
8. Zhai, L., Chaturvedi, D. & Cumberledge, S. Drosophila wnt-1 undergoes a hydrophobic modification and is targeted to lipid rafts, a process that requires porcupine. J Biol Chem 279, 33220-33227 (2004).
9. Kurayoshi, M., Yamamoto, H., Izumi, S. & Kikuchi, A. Post-translational palmitoylation and glycosylation of Wnt-5a are necessary for its signalling. Biochem J 402, 515-523 (2007).
10. Franch-Marro, X. et al. In vivo role of lipid adducts on Wingless. J Cell Sci 121, 1587-1592 (2008).
11. Steinhauer, J. & Treisman, J. E. Lipid-modified morphogens: functions of fats. Curr Opin Genet Dev 19, 308-314 (2009).
12. Nusse, R. Wnts and Hedgehogs: lipid-modified proteins and similarities in signaling mechanisms at the cell surface. Development 130, 5297-5305 (2003).
13. Mann, R. K. & Beachy, P. A. Novel lipid modifications of secreted protein signals. Annu Rev Biochem 73, 891-923 (2004).
14. Niehrs, C. Regionally specific induction by the Spemann-Mangold organizer. Nat Rev Genet. 5, 425-434 (2004).
15. Petersen, C. P. & Reddien, P. W. Wnt signaling and the polarity of the primary body axis. Cell 139, 1056-1068 (2009).
16. He, X., Semenov, M., Tamai, K. & Zeng, X. LDL receptor-related proteins 5 and 6 in Wnt/beta-catenin signaling: arrows point the way. Development 131, 1663-1677 (2004).
17. Kiecker, C. & Niehrs, C. A morphogen gradient of Wnt/beta-catenin signalling regulates anteroposterior neural patterning in Xenopus. Development 128, 4189-4201 (2001).
18. McGrew, L. L., Hoppler, S. & Moon, R. T. Wnt and FGF pathways cooperatively pattern anteroposterior neural ectoderm in Xenopus. Mech Dev 69, 105-114 (1997).
19. Resh, M. D. Palmitoylation of ligands, receptors, and intracellular signaling molecules. Sci STKE 2006, re14 (2006).
20. Linder, M. E. & Deschenes, R. J. Palmitoylation: policing protein stability and traffic. Nat Rev Mol Cell Biol 8, 74-84 (2007).
21. Zeidman, R., Jackson, C. S. & Magee, A. I. Protein acyl thioesterases (Review). Mol Membr Biol 26, 32-41 (2009).
22. Duncan, J. A. & Gilman, A. G. A cytoplasmic acyl-protein thioesterase that removes palmitate from G protein alpha subunits and p 21(RAS). J Biol Chem 273, 15830-15837 (1998).
23. Camp, L. A. et al. Molecular cloning and expression of palmitoyl-protein thioesterase. J Biol Chem 269, 23212-23219 (1994).
24. Verkruyse, L. A. & Hofmann, S. L. Lysosomal targeting of palmitoyl-protein thioesterase. J Biol Chem 271, 15831-15836 (1996).
25. MacDonald, B. T. et al. Wnt signal amplification via activity, cooperativity, and regulation of multiple intracellular PPPSP motifs in the Wnt co-receptor LRP6. J Biol Chem 283, 16115-16123 (2008).
26. Wan, J., Roth, A. F., Bailey, A. O. & Davis, N. G. Palmitoylated proteins: purification and identification. Nat Protoc 2, 1573-1584 (2007).

### Example 9

### Tiki2−/− Mice Exhibit High Bone Mass

Unlike all other mammals including humans, the rodent lineage including the mouse only has the Tiki2 gene. One possibility, which we speculate, is that Tiki1 has a restricted role in early embryonic patterning in vertebrates including Xenopus as we have demonstrated, and during evolution the rodent lineage somehow lost Tiki1 due to its unique ‘egg cylinder’ stage that does not occur in other vertebrates/mammals. This situation gave us an opportunity to create mutant mice that lack any Tiki activity. Surprisingly homozygous Tiki2−/− null mice are viable and fertile, and are found at the expected Mendelian frequencies on a mixed genetic background. Tiki2−/− mice appear overtly normal and do not display obvious neurological or behavioral phenotypes. A lack of overt developmental phenotypes has been frequently observed in Wnt antagonist mutant mice (Satoh et al. Genesis. 2008. 46:92-103; Furushima et al. Dev Biol. 2007. 306:480-92; Kansara et al. J Clin Invest. 2009. 119:837-51) due likely to functional redundancy.

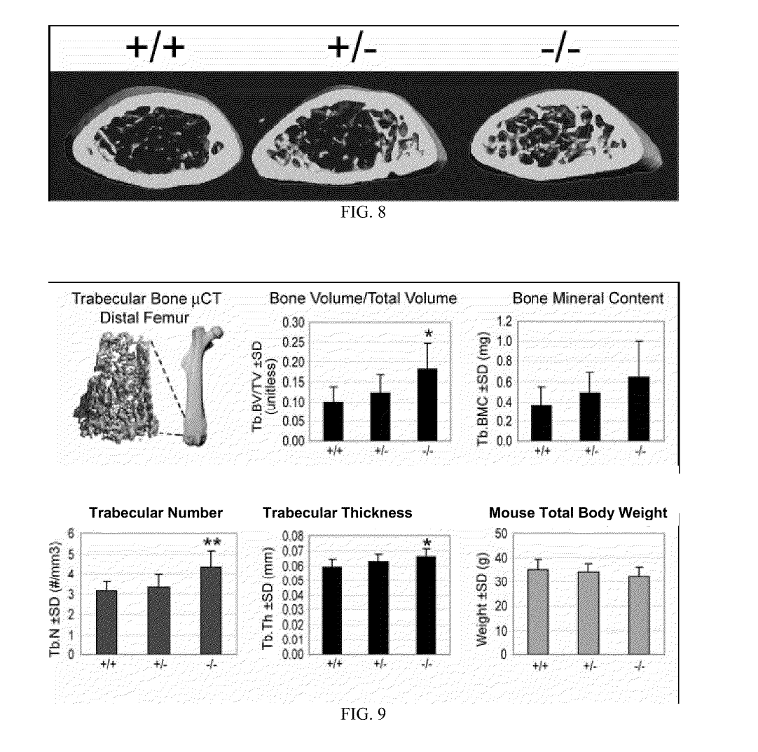

Given the importance of the Wnt pathway in regulating bone mass in mice and human, we evaluated Tiki2−/− mice for a possible bone phenotype. Expression array studies (Latin et al. Immunome Res. 2008. 4:5) indicate that mouse Tiki2 is highly expressed in osteoblasts but not osteoclasts (not shown). 16-week old mice were analyzed by μLCT, and a greater amount of trabecular bone was found in Tiki2−/− mice (FIG. 8). Quantification of the trabecular bone properties revealed a significant increase in bone volume fraction, trabecular number and trabecular thickness in Tiki2−/− mice in comparison with +/+ littermates (FIG. 9), while no significant changes were found in femur length or total body weight (FIG. 9). Although the difference between the WT and Tiki2+/− mice did not reach to a statistically significant level, the average values for the Tiki2+/− heterozygotes were consistently higher and thus may suggest a gene dosage dependent effect (FIG. 9). It should be noted that these mice were from a mixed genetic background, which likely contributed to some intra-sample variations. Based on the relative level of increase and previous work by others and us with the Dkk1+/− mouse (MacDonald et al. Bone. 2007. 41:331-9; Morvan et al. J Bone Miner Res. 2006. 21:934-45), it appears that the Tiki2−/− mutants have a comparatively larger increase in key measurements (such as BV/TV).

By this comparison we anticipate that the Tiki2−/− bone phenotype might be more prominent than some other published Wnt inhibitor mutants (Ellwanger et al. Mol Cell Biol. 2008. 28:4875-82; Bodine et al. Mol. Endocrinol. 2004. 18:1222-37). These preliminary and ongoing analyses suggest that Tiki2 is likely a key negative regulator of bone mass. Given that mice and human share indistinguishable mechanisms for bone mass regulation, particularly with regard to Wnt/LRP5 signaling, it is most likely that Tiki2 negatively regulates bone mass in human.

### Example 10

### HTS for Tiki2 Inhibitors

A high throughput screen (HTS) is used to identify compounds that enhance LRP5/6 signaling as potential therapeutics for osteoporosis or cellular proliferative disorders. We employ a Wnt responsive cell line carrying a Super-TOPFLASH(STF) luciferase reporter (Wnt-responsive) (Major et al. Science. 2007. 316:1043-6). These STF cells grow as an adherent monolayer making them ideally suited for 384-well-plate screening format. In addition, these STF cells possess a low level of basal Wnt signaling that can be readily induced by the addition of Wnt3a, which in a 6-hour treatment resulted in 30-40 folds induction of the STF reporter (not shown). Different conditions are tested and an acceptable z′ score of 0.5 or above (z′ measures HTS quality and reproducibility (Zhang et al. J Biomol Screen. 1999. 4:67-73)) is achieved.

To screen for Tiki1/2 inhibitors, we will first establish clonal lines of the above STF cells that express Wnt3a (which we will refer to as STF-Wnt3a) using a retroviral Wnt3a expression vector. We will select individual clones showing the highest levels of luciferase activity while preserving adherent monolayer growth. Next we will establish clonal lines of STF-Wnt3a cells expressing Tiki1 and/or 2 via another retroviral vector (under different selection). Lastly we will confirm that Tiki1/2 shRNA knockdown in our STF-Wnt3a/Tiki2 cells will lead to derepression of the STF luciferase reporter, mimicking the effects of a potential small molecule Tiki1/2 inhibitor. These multiple steps ensure that we have STF cells responsive to Wnt3a, which is inactivated by Tiki2, which is further inhibitable by potential compound inhibitors. Importantly because our assay is based on increases of STF reporter expression, non-specific compounds that cause cell toxicity/death are excluded by definition.

A pilot scale screen is conducted treating STF-Wnt3a/Tiki2 cells with the standard 10 μM compounds for 6 or 12 hrs to optimize the screening condition. According to our and others' experience a ratio of about 0.5% positive hits is reasonable. Once we have established the optimal conditions, we will screen the entire ICCB chemical library collection through triplicate readings for each compound.

After the primary screen and subtractions of compounds that are not specific for Tiki2, we select compounds secondary confirmation screens. We perform a dose response curve of each compound (1 nM to 1 μM) using the same STF-Wnt3a/Tiki2 cells in 384-well plate format. The compounds that successfully activate STF-Wnt3a/Tiki2 in the confirmation assay are tested further in immunostaining for nuclear β-catenin, which is induced by Wnt3a in these STF cells (Major et al. Science. 2007. 316:1043-6). Thus STF-Wnt3a/Tiki2 cells will have low nuclear β-catenin, and inhibition of Tiki2 will restore/increase nuclear β-catenin These assays are performed in 384-well plate format using the high-content imaging HTS system at ICCB. These assays together will allow the selected of the strongest candidates, which are used to demonstrate whether these compounds inhibit Tiki2 directly. Because Tiki2 causes Wnt3a to exhibit faster mobility in electrophoresis (FIG. 5C) and to partition in aqueous phase (FIG. 6A,B), Tiki2 inhibitors should restore Wnt3a mobility and partitioning in detergent phase. These secondary screens together identify potent and functional Tiki2 inhibitor compounds.

Further functional validation of Tiki inhibitors is done as follows. Once we identify candidate Tiki inhibitors, we evaluate their specificity. For example, we confirm that these compounds will not affect Sclerostin or DKK1 inhibition of Wnt3a. We also examine whether these compounds affect other signaling pathways, such as Hedgehog, TGFβ, BMP, and EGF/FGF. These can be performed using pathway-specific luciferase reporters and/or phospho-Abs (for Smad, MAPK, etc) in mammalian cell lines.

Once we establish the specificity of candidate Tiki2 inhibitors, we test these compounds in primary osteoblasts from WT and Tiki2−/− mice. Because Tiki2 is expressed in osteoblasts and Tiki2−/− mice exhibit high bone mass, Tiki2−/− osteoblasts may proliferate or differentiate into bone more robustly than the WT ones. Ideally, Tiki2 inhibitors will mimic the effects of Tiki2 deletion, and further, these compounds should have little or no effects on Tiki2−/− osteoblasts. These experiments further demonstrate the specificity of the compounds and may validate their potential in stimulating osteoblast proliferation/differentiation in vitro. We also examine the effect of these Tiki2 inhibitors in human Saos-2 cells, which are Wnt-responsive and are commonly used for their osteoblast-like properties (Suzuki et al., J Cell Biochem. 2008. 104:304-17). We anticipate that Tiki2 inhibition leads to increased proliferation/differentiation of these cells. These studies are compared to those using primary osteoblasts from WT and Tiki2−/− mice, substantiating similar regulation of osteoblast properties by Tiki2 between mice and men.

The most promising Tiki2 inhibitors are tested in mice. The fact that Sclerostin blocking Abs promote bone formation in an aging rat model (Li et al., J Bone Miner Res. 2009. 24:578-88) implies that Wnt stimulation of bone growth continues into late adulthood. Thus Tiki2 inhibition, and thereby production of more active Wnts, will likely benefit bone mass throughout life. We compare the effect of Tiki2 inhibitors in mice with the results from our Tiki2 deletion mutants. Tiki2 compound inhibitors that increase bone mass in mice are selected for further therapeutic development.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of identifying a candidate compound for maintaining or increasing bone density or for treating a cellular proliferative disorder, the method comprising:
providing a cell that is responsive to Wnt3A that expresses Tiki1 or Tiki2;
contacting the cell with a test compound;
evaluating an effect of the test compound on signaling in response to Wnt3A in the cell,
identifying as a candidate compound a test compound that inhibits signaling in response to Wnt3A in the cell.

2. The method of claim 1, further comprising determining that the candidate compound acts specifically on Tiki1 or Tiki2.

3. An isolated Tiki1N or Tiki2N polypeptide.

4. An isolated nucleic acid encoding the Tiki1N or Tiki2N polypeptide of claim 3.

5. A vector comprising the nucleic acid of claim 3.

6. The vector of claim 5, further comprising one or more regulatory sequences operatively linked to the Tiki1N/2N nucleic acid sequence.

7. A host cell expressing the vector of claim 5.

8. An antibody or antigen binding fragment thereof that specifically binds a Tiki1 or Tiki2 polypeptide.

9. A transgenic animal, whose somatic and germ cells have:
(i) an exogenous recombinant Tiki1 or Tiki2 nucleic acid molecule stably integrated into the genome of said mammal, or
(ii) a disrupted Tiki1 or Tiki2 gene.

10. A cell derived from the transgenic animal of claim 9.

11. A method of treating a disorder associated with increased Wnt signaling, the method comprising administering a therapeutically effective amount of a composition comprising:
(ii) Tiki1 or Tiki2 polypeptide, or a variant or active fragment thereof;
(ii) Tiki1N or Tiki2N polypeptide, or a variant thereof;
(iii) a nucleic acid encoding a Tiki1 or Tiki2 polypeptide, or a variant or active fragment thereof; or
(iv) a nucleic acid encoding a Tiki1N or Tiki2N polypeptide, or a variant thereof.

12. The method of claim 11, wherein the disorder is associated with unwanted cellular proliferation.

13. The method of claim 11, wherein the disorder is cancer.

14. A method of treating a disorder associated with reduced Wnt signaling, the method comprising administering a therapeutically effective amount of an inhibitory nucleic acid that specifically decreases expression of Tiki1 or Tiki2.

15. The method of claim 14, wherein the disorder is associated with bone loss or resorption.

16. The method of claim 15, wherein the disorder is osteoporosis.

17. The method of claim 14, wherein the inhibitory nucleic acid is selected from the group consisting of siRNA, antisense, ribozymes, and aptamers.


SEQUENCE LISTING (13 sequences)

SEQ ID NO: 1 - protein, 505 aa, Homo sapiens
MSPWSWFLLQTLCLLPTGAASRRGAPGTANCELKPQQSELNSFLWTIKRDPPSYFFGTIHVPYTRVWDFIPDNSKEAFLQSSIVYFELDLTDPYTISALTSCQMLPQGENLQDVLPRDIYCRLKRHLEYVKLMMPLWMTPDQRGKGLYADYLFNAIAGNWERKRPVWVMLMVNSLTEVDIKSRGVPVLDLFLAQEAERLRKQTGAVEKVEEQCHPLNGLNFSQVIFALNQTLLQQESLRAGSLQIPYTTEDLIKHYNCGDLSSVILSHDSSQVPNFINATLPPQERITAQEIDSYLRRELIYKRNERIGKRVKALLEEFPDKGFFFAFGAGHFMGNNTVLDVLRREGYEVEHAPAGRPIHKGKSKKTSTRPTLSTIFAPKVPTLEVPAPEAVSSGHSTLPPLVSRPGSADTPSEAEQRFRKKRRRSQRRPRLRQFSDLWVRLEESDIVPQLQVPVLDRHISTELRLPRRGHSHHSQMVASSACLSLWTPVFWVLVLAFQTETPLL

SEQ ID NO: 2 - protein, 517 aa, Homo sapiens
MHAALAGPLLAALLATARARPQPPDGGQCRPPGSQRDLNSFLWTIRRDPPAYLFGTIHVPYTRVWDFIPDNSKAAFQASTRVYFELDLTDPYTISALASCQLLPHGENLQDVLPHELYWRLKRHLDYVKLMMPSWMTPAQRGKGLYADYLFNAIAGNWERKRPVWVMLMVNSLTERDVRFRGVPVLDLYLAQQAEKMKKTTGAVEQVEEQCHPLNNGLNFSQVLFALNQTLLQQESVRAGSLQASYTTEDLIKHYNCGDLSAVIFNHDTSQLPNFINTTLPPHEQVTAQEIDSYFRQELIYKRNERMGKRVMALLRENEDKICFFAFGAGHFLGNNTVIDILRQAGLEVDHTPAGQAIHSPAPQSPAPSPEGTSTSPAPVTPAAAVPEAPSVTPTAPPEDEDPALSPHLLLPDSLSQLEEFGRQRKWHKRQSTHQRPRQFNDLWVRIEDSTTASPPPLPLQPTHSSGTAKPPFQLSDQLQQQDPPGPASSSAPTLGLLPAIATTIAVCFLLHSLGPS

SEQ ID NO: 3 - protein, 420 aa, Caenorhabditis elegans
MTFYILVVSLYLSLFLVTVVQSDCDTDVEQRERNIFLWSVKHPQFASSQGYLFGTIHVPFTEVWKEVSDRVRDAFAVSDTVLLEIDLHDEATIHELIACKNLAYDETVHSYLSIELLERIEKIMEYLRSSFLAWAQKQNPRDTKKIKHAEDIYNNIIGDWWRKRPIWLLFLLYQMCENVFEKSSSPLLDLYIAQRATDEKKTIIPIETAEEQCNPVVSVSTNEIIFAIEHTVHYFEDKILDNPSKDNESRSSLKELVEHYKCGTLKEDMFDKDGMSIIDYATGTTERFKADEINKKLKQDIFVKRNLRMAKRIEKILKGRNSNTVFSAIGAGHFFGSSSVLTYLEESGFIVQKLKNTDVIQPLRSPYRQTAKFKRVWTKETAVRRKSIIIEEVAPSSSRIARLWLVPCIFLLHSIFAIFP

SEQ ID NO: 4 - protein, 508 aa, Xenopus tropicalis
MVIIWNIFLPAFLLVLAKASLRSSRDSANCKLNKKQSQLNSFLWTIKRDPPSYFFGTIHVPYTRVWDFIPENSKTAFQQSNIVYFELDLTDPYTISALTSCQMLPQGENLQNVLPRDIYRRLKRHLEYVKLMMPSWMTPDQRGKGLYADYLFNAIAGNWERKRPVWVMLMVNSLTEVDIKSRGVPVLDLYLAQEAERLKKRTGAVEQVEEQCHPLNGLNLSQVIFALNQTLLQQENLRAGSVQVPYSTEDLIKHYNCGDLNSIIFNHDSSQVPNFINSTLPPQERITAQEIDNYFRQELIYKRNERMGKRVKDLLEQFPEKSFFFAFGAGHFLGNNTVIDVLKRYGYDVLHTPAGRSIINNGKGKKNLLPSKFSSSSLPVGLSAPPCTVTSRIKQSINSHKDQESLPDILLDDDIDQLDKDERKYKKRKQRKEKHRHFSDLWVRIQESSTDTTPQIRIINGYITVEPHPREHGKDKYIKAAQSVSFSLSIPSAFLLLAWCFQQVAVLQ

SEQ ID NO: 5 - protein, 523 aa, Xenopus tropicalis
MGKTMWARAVFLCFSVGTLLWQEVLTRRIPVDTGQCGLPKSQEDLNSFLWTVRRHPPAYLFGTIHVPYTRVWDFIPQNSKKAFHDSNSVYFELDLTDPYTISALANCQMLPQGENLQDVLPRDLYRRLKRHLEYVKHMMPHWMTPDQRGKGLYADYLFNAIAGNWERKRPVWVMLMVNSLTEADIRSRGVPVLDLYLAQEADRMKKKTGAVERVEEQCHPLNRLNLSQVLFALNQTLLQHESLRAGSFQAPYTTEDLIKHYNCGDLNAVIFSHDSSQLPNFINVTLPPHEQVTAQEIDIYFRQELIYKRNERMARRVIALLKENKDKSFFFAFGAGHFLGNNTVIDVLRQNGYEVEHTPAGQTFTAAKPKTNPTSDDSMATDSPAMKYFDHVPATASYFGESDEEMLPPHLLLPDSISQLEEFGKQNSWHRKHYRNQRPRQFNDLWVRLDDSTTTLPSNTRNTNGEQSAESLVWLPEQDHHNYLDVKLSHSQSNSSPKCLSASPAFLYTLVTLCLITTMRTRS

SEQ ID NO: 6 - protein, 500 aa, Danio rerio
MTMMTMMMVSWSAFLQICWILMVRANQFNPGEPSGCRTNTPQSDLNSFLWTIKRDPPSYLYGTIHVPYTRVWDFIPQNSKQAFQESSVVYFELELTDPSTISALSRCQLLPAGQNLQDVLPPELYLRLKTHLEYVRLMLPSWMTPDQRGKGLYAEYLFNAIAGNWERKRPVWVMLMVNSLTEADIKTRGVPVLDLYLAQEAERMKKQTGAVEKVEEQCSPLNTLDFSQVIFALNQTLLQQESVRAGSLQVPYTTEHLITHYNCGDLHSIISHDTAQVPNFNNVTLRPSDQVTAQQIDSYFRRELIYKRNERMGRRVTALLQEQPHKTFFFAFGAGHFLGNNSVIDVLRREGYEVEHTPAGQPLHRRSGWRSADPADTDAPADTDAALQPFLHHSRHHELQLLEGLELLEKVEHKLKKKHRRNKLKKQRQFNDLWVRMEDSVTAEAPPPLIHIINGYITVQTHPQEHERANHDRTFSGSSSWTGPALSALAVCVQMLRLLL

SEQ ID NO: 7 - protein, 520 aa, Danio rerio
MNCQSGLRWLVTLCAFFQVGSARDTHESTRQCDKPVSQKDMNSFLWTVKRPRPFPPSYLFGTIHVPYTRVWDYIPESSKRAFQTSNSVFFELDLTDPLTISKLTSCQLLPNGENLQTLLPRDLYRRLKRHLDYVKHMMPFWMTADQRGRGLYADYLFNAIAGNWERKRPVWVMLMVNSLTEADVRSRGTPVLDLFLAQEAERLGKQTGAVERVEEQCHPLNGLNFSQVLFALNQTLLQHESLRAGILQGTFTTEDLIAHYNCGDLNSIIFNHDTSQLPHFINSSLPDHERLTAQQIDSYLRQELIYKRNERMARRVSALLQRNPNQSFFFAFGAGHFLGNHSVLDILRQEGYEVEHTPPQEPIIQSWSEREATTLNPTEDSFESVSVTEWTSETPELEEISQEELSHMLLPDSLSQLEEFGRYKHPRKTHHTHSRPRLFSDLWVRIGDSTTPHPSIRITNGYVTVEPPQIRQEQQQRLRERLKPLSEPTNPSALDSAAPNPTYALTCFLACLISQLLFAS

SEQ ID NO: 8 - protein, 328 aa, Nematostella vectensis
QPQKNFNSFLWLVKRTPPAYFYGTIHVPYTRVWDFIPMNSKQAFTASQHVYFELDLTDEKTMRALMKCQMLPSGTMLRQTLPRKMFKRLKSHLRYIKRMIPKWIKHRDQETSSAGPYANKLYEMLTKDWDKKRPIWVMLMVNSLTESDIKTRGIPVLDQYLALEASRNHKLIGAVENVDEQCKPLNALNASQVVFALNQSLHFQERLRRGQVQVTYTTDDLIDHYNCGDLKSVLFSTQTSLPTLTVNSSLEQRERKRAQEIDQYFRNELIFQRNKRMAQRVITLLNNHPEKDFFFAFGAGHFLGNHSIIDIMKKHGYDVEYVKPEQEL

SEQ ID NO: 9 - DNA, 1733 bp, Homo sapiens
ccccgcggccccggctagccagggcgggcggccacctctgccctactccttccctccgcgttccgggcctcggagccgccttgaggaggatgagtccctggagctggttcctgctgcagaccctctgcctcctgcccacgggcgcagcttcgcggcgcggggcgcccggcaccgccaactgcgagctcaagccccaacaaagcgagctgaattccttcttgtggaccattaagcgagacccaccatcttacttctttggcacaatccatgtcccgtacacccgagtttgggacttcatccccgacaactctaaggaggctttcctgcagagcagcattgtgtactttgagttggatctcacagacccctataccatctcagctctcaccagctgtcagatgctgccacagggcgagaacctccaagatgtgctccccagggacatctactgccgcctcaagcgccacctggagtatgtcaagctcatgatgcccttgtggatgaccccagaccagcgcggcaaggggctctacgcagactacctcttcaatgctattgccggaaactgggagcgcaagaggcctgtctgggtgatgctcatggtcaactccctgactgaagtggacattaagtcccgtggagtgcctgtcttagacctgttccttgcccaggaggctgagcggctgaggaaacagactggggcagtggaaaaggtggaagagcagtgccatccattgaatgggttgaacttttcacaggttcccaattttattaatgccacgctaccacctcaggagcgcatcactgctcaggagattgacagctacttacgccgggagctgatctacaagcggaatgagagaatagggaagcgggtgaaggcccttttggaggagttccctgacaaaggcttcttctttgcctttggagctggtcatttcatgggcaacaacacagtgctggatgttttgcggcgtgaaggctatgaggtagaacacgcccctgctggacgacccatccacaaagggaagagtaaaaagacctccacacggcccactctgtccaccatctttgctccaaaagtccctaccctggaagtaccggcaccagaagccgtatcctcagggcactcaacgctgcctccccttgtgtcccggcctggaagtgccgacacgcccagtgaggccgaacagaggttccggaagaagcggaggcggtcacagcggaggccgcgactccggcaattcagcgatctgtgggtccgcctggaggagagtgacatagtcccgcaactccaggtccctgtcctggacaggcacatctccactgaactgcggctccctcgccgtgggcattcccaccacagccagatggtggccagcagtgcctgcctgtctctctggactcctgtgttctgggtgctggtgctggctttccaaacagagacacccctcctgtaacgactggaagcaccaggctaagaacctgacccctcggacttgaagaatggccattcctgtactccacattctggtctagccttgttgggcccaatccagaagagactgctcttgaaaaagcggcccagtgttgattcttctctttccaaggaatgtgactttgggttatccaactttgggggcaggtgtacagttttgtaacatagtgagttgtgtgaaaataaattataaatgagttgtatgaaaaaaaaaaaaaaaaaaaaaaaaaaaaaa

SEQ ID NO: 10 - DNA, 1819 bp, Homo sapiens
atgcacgccgccctggcggggccgctgctcgccgccctcctcgccaccgctcgcgcccgcccgcagcccccggacggaggacagtgccggccgcccggatcgcagagggacttgaactccttcctgtggacgattcggcgtgatcctccggcctacctgtttggcactattcacgtcccctacacccgcgtctgggacttcatcccggacaactccaaggcagccttccaggctagcacccgtgtctactttgagctggaccttacagacccctacaccatctcggccctggccagctgccagctgctgccgcacggggaaaacctgcaggacgtgctgccccacgagctttactggcgcttgaagcgccacctggactacgtgaagttgatgatgccctcctggatgacgcccgctcagcggggcaaggggctctatgctgactacctattcaatgccatcgcgggcaactgggagcgcaagaggcccgtctgggtgatgctcatggtaaactcgctcacagagagggacgtgcgcttccgtggtgtgcccgtgctcgacctctacctggcccagcaggctgagaagatgaagaagaccacaggggctgtggagcaggtggaggagcagtgccatcccctcaacaacgggctcaacttctcccaggtgctgtttgccctgaaccaaaccctgctgcagcaggagagtgtgcgggccgggagcctgcaggcctcctacaccacggaggacctcatcaagcactacaactgcggagacctcagcgcagtcatcttcaaccacgacacatcccagctgcccaactttatcaacaccaccctcccgccacacgagcaggtgacggcccaggagattgacagctacttccgccaggagctcatctacaagaggaatgagcgcatggggaagagggtcatggcgcttctacgggagaacgaggacaagatctgcttctttgccttcggagcaggtcactttctggggaacaacacagtcatcgacatcctgcggcaggcagggctggaggtggaccacacacccgccgggcaggccatacacagccctgccccccagagcccagcgccctctcctgaggggacctcgacgagcccggccccagtgaccccagctgccgctgtccccgaagcaccctctgtgacccccaccgccccaccagaggatgaggatccagccctgtccccacacctcctgctccccgacagcctcagccagctggaggagtttggccggcagaggaagtggcacaagaggcagagcacacaccagcggccgcggcagttcaatgacctctgggtccgcatcgaggacagcaccaccgcctcaccacccccgctgcccctgcagcccacccacagctcggggaccgccaagccccccttccagctttcagaccagctacaacagcaggacccgccagggcccgccagcagctcggcacccaccctgggccttctccccgccatcgccaccaccatcgctgtctgcttcctgctgcatagccttgggccctcctgacctcggccaccccaagtggagaagccagagaaatgacacaggggtcttcggccacacccaactgctcctgccaccacctccagggggtgcccaacacgtcgagggcagtccacatgggattggctagacactctagagctttactgtacccaagttacatcttcttttttgtagaaggatcttaatttcccatagtgctatggggctcttttgtaaaatacgtgtccatattaaaaaagaaaaatgtatttattctaccgataaa

SEQ ID NO: 11 - protein, 481 aa, Artificial Sequence
  Recombinant protein
MSPWSWFLLQTLCLLPTGAASRRGAPGTANCELKPQQSELNSFLWTIKRDPPSYFFGTIHVPYTRVWDFIPDNSKEAFLQSSIVYFELDLTDPYTISALTSCQMLPQGENLQDVLPRDIYCRLKRHLEYVKLMMPLWMTPDQRGKGLYADYLFNAIAGNWERKRPVWVMLMVNSLTEVDIKSRGVPVLDLFLAQEAERLRKQTGAVEKVEEQCHPLNGLNFSQVIFALNQTLLQQESLRAGSLQIPYTTEDLIKHYNCGDLSSVILSHDSSQVPNFINATLPPQERITAQEIDSYLRRELIYKRNERIGKRVKALLEEFPDKGFFFAFGAGHFMGNNTVLDVLRREGYEVEHAPAGRPIHKGKSKKTSTRPTLSTIFAPKVPTLEVPAPEAVSSGHSTLPPLVSRPGSADTPSEAEQRFRKKRRRSQRRPRLRQFSDLWVRLEESDIVPQLQVPVLDRHISTELRLPRRGHSHHSQMVASS

SEQ ID NO: 12 - protein, 494 aa, Artificial Sequence
  Recombinant protein
MHAALAGPLLAALLATARARPQPPDGGQCRPPGSQRDLNSFLWTIRRDPPAYLFGTIHVPYTRVWDFIPDNSKAAFQASTRVYFELDLTDPYTISALASCQLLPHGENLQDVLPHELYWRLKRHLDYVKLMMPSWMTPAQRGKGLYADYLFNAIAGNWERKRPVWVMLMVNSLTERDVRFRGVPVLDLYLAQQAEKMKKTTGAVEQVEEQCHPLNNGLNFSQVLFALNQTLLQQESVRAGSLQASYTTEDLIKHYNCGDLSAVIFNHDTSQLPNFINTTLPPHEQVTAQEIDSYFRQELIYKRNERMGKRVMALLRENEDKICFFAFGAGHFLGNNTVIDILRQAGLEVDHTPAGQAIHSPAPQSPAPSPEGTSTSPAPVTPAAAVPEAPSVTPTAPPEDEDPALSPHLLLPDSLSQLEEFGRQRKWHKRQSTHQRPRQFNDLWVRIEDSTTASPPPLPLQPTHSSGTAKPPFQLSDQLQQQDPPGPASSSAPT

SEQ ID NO: 13 - DNA, 25 bp, Artificial Sequence
  Synthetically generated oligonucleotide
ccaaatgattaccatcatagctcag